(19) United States
(12) Patent Application Publication
(10) Pub. No.: US 2026/0365958 A1
(43) Pub. Date: Nov. 19, 2026

(54) METHODS FOR LARGE-SCALE SPHEROIDS AND EXTRACELLULAR VESICLES PRODUCTION

(71) Applicant: CORNING INCORPORATED, CORNING, NY (US)

(72) Inventors: Samantha Sophie Catherine Haller, Paris (FR); David Henry, Fontaine le Port (FR)

(21) Appl. No.: 19/129,993

(22) PCT Filed: Nov. 20, 2023
(86) PCT No.: PCT/US2023/080478

§ 371 Date: May 14, 2025

(30) Foreign Application Priority Data
Nov. 21, 2022 (US) 63426908

(51) Int. Cl. C12M 1/00; C12M 1/12; C12N 5/00

(52) U.S. Cl. CPC C12M 29/10; C12M 25/14; C12M 29/18; C12N 5/0062; C12N 2513/00; C12N 2533/74; C12N 2537/10; C12N 2539/00

(57) ABSTRACT

A method for the production of spheroids and cellular byproducts of spheroids such as extracellular vesicles is provided. The method includes providing a bioreactor having a cavity for culturing cells; inserting cells into the cavity to form spheroids; and perfusing a cell culture medium through the cavity to culture the spheroids. The porous scaffold may be dissolvable or non-dissolvable, and the cells are seeded into the pores of the porous scaffold, where the cells aggregate in the pores of the porous scaffold to form spheroids. The bioreactor is continuously perfused for large scale spheroid production and enables harvesting of spheroids and/or extracellular vesicles or other elements from the cell secretome.

### CROSS-REFERENCE TO RELATED APPLICATIONS

This application claims the benefit of priority under 35 U.S.C. § 119 of U.S. Provisional Application Ser. No. 63/426,908 filed on Nov. 21, 2022, the content of which is relied upon and incorporated herein by reference in its entirety.

### FIELD

The present disclosure generally relates to methods for producing spheroids and/or byproducts of cells, including extracellular vesicles. In particular, the present disclosure relates to methods of large-scale production of spheroids and/or extra-cellular vesicles in porous scaffolds.

### BACKGROUND

In vitro models of cellular function have been developed in various fields like cancer and stem cell research, drug discovery and drug screening, and regenerative medicine, among others. The goal of these models is to understand at the molecular and cellular level of how these cells function and interact. These in vitro models were developed using two-dimensional (“2D”) systems. Although these 2D systems have achieved significant discoveries in these fields, many gaps remain. With 2D systems, cells adhere to rigid surfaces and are geometrically constrained, leading to a flat morphology, which alters the cytoskeleton regulation that is important in intracellular signaling, and consequently can affect cell growth, migration, and apoptosis. These limitations of 2D cultures often result in biological responses in-vitro that are strikingly different from what is observed in-vivo. This is because these 2D models fail to fully capture the in vivo tissue complexity and the contribution of the cellular and non-cellular microenvironment.

Currently, in drug discovery, the standard procedure of screening compounds starts with 2D cell culture-based tests, followed by animal model tests, and then clinical trials. Only about 10% of the compounds progress successfully through clinical development. Many drugs fail during clinical trials (especially during phase III, which is the most expensive phase of clinical development) largely due to the lack of clinical efficacy and/or unacceptable toxicity. A portion of these failures is attributed to data collected from the 2D culture tests in which the cellular response to drug(s) is altered due to their unnatural microenvironment. Due to the high costs associated with drug discovery, demand has risen for the ability to dismiss ineffective and/or unacceptable toxic compounds as early in the drug discovery process as possible. In-vitro cell-based systems that can more realistically mimic the in-vivo cell behaviors and provide more predictable results for in-vivo tests are needed.

Of particular interest are three-dimensional (“3D”) systems. 3D cell cultures provide enhanced cell-cell interactions that more closely mimic the natural microenvironment of a tissue as compared to 2D cell culture monolayers. In the last decade, 3D cell cultures have been used for growing a wide variety of cancerous and non-cancerous cell lines into spheroids or 3D cell colonies. Spheroids have been used in 3D tissue modeling in the fields of drug discovery, toxicology, and regenerative medicine. Recent research suggests that 3D cell culture, in contrast to 2D culture, more accurately represents the environment experienced by cells in-vivo, and that cell responses in 3D cultures are more similar to in-vivo behavior than the cell responses in 2D cultures. The additional dimensionality of 3D cultures is believed to lead to the differences in cellular responses because not only does it influence the spatial organization of the cell surface receptors engaged in interactions with surrounding cells, but it also induces physical constraints to cells. These spatial and physical aspects in 3D cultures are believed to affect the signal transduction from the outside to the inside of cells, and ultimately influence gene expression and cellular behavior.

Cell culture systems are also of interest for elements produced by cells and secreted in their environment. One element of particular interest (especially regarding regenerative medicine and other therapies) is extracellular vesicles. Extracellular vesicles (“EVs”) are nanoparticles produced by most cell types, and they have a strong therapeutic potential. Current methods used for EV production use adherent cells either on a 2D surface requiring large culture space to meet the concentration needed for therapeutic or on a 3D surface like microcarriers or other fixed bed material under perfusion in a bioreactor. However, methods for EV production that are on a large scale and efficient remain elusive despite there being a need to mass produce this important byproduct of cells.

Cells that aggregate into 3D structures are called spheroids, and spheroids better mimic in vivo tissues than cells grown in 2D. Creating large quantities of spheroids is advantageous for a variety of reasons, including for testing of drugs and for acting as cellular factories for producing cell byproducts such as EVs and exosomes. As EVs are a cellular byproduct, they can be collected from fluid that surrounds cells and cell aggregates like spheroids. But where drug testing on cell types is desired (or when other applications such as research about cell function or regenerative medicine is desired), capturing spheroids themselves in large quantities is advantageous. Spheroids are presently grown on 2D platforms such as microwell plates and flasks. However, the 2D nature of these platforms limit the number of spheroids generated. Further, as EVs are produced by the cells in the spheroids, the limited numbers of spheroids generated by 2D systems therefore also limits the amount of EVs that can be produced.

Capturing spheroids from 2D systems (harvesting them) is labor intensive and inefficient as each plate will have to be processed individually to collect spheroids from microwells, and growing spheroids in 2D systems often requires liquid exchange, making it difficult to culture the spheroids without disturbing them. Likewise, capturing EVs and other cellular byproducts produced by the spheroids is also labor intensive and inefficient as liquid has to be collected and separated from the spheroids.

Accordingly, there is an ongoing need for 3D matrices that allow for 3D cell culture to produce spheroids and/or cellular byproducts (for example, extracellular vesicles) on a large scale. It is further advantageous to have adequate delivery of nutrients to the cells being cultured and also an ultimate harvesting or recovery of the cultured cells and/or cellular byproducts. The present disclosure provides a 3D porous scaffold for culturing spheroids and cellular byproducts produced by the spheroids that solves the above problems encountered with 2D culturing.

### SUMMARY

According to aspects of the present disclosure, a method for cell spheroid production is provided herein. The method includes providing a bioreactor having a cavity for culturing cells, inserting cells into the cavity to form spheroids, and perfusing a cell culture medium through the cavity to culture the spheroids. Aspects of embodiments include the bioreactor including a porous scaffold in the cavity. The porous scaffold is made of interconnected scaffold material defining pores. In certain aspects, the porous scaffold is a foam scaffold that in some embodiments is dissolvable, and in other embodiments is non-dissolvable. The method can further include, by inserting the cells into the cavity, seeding the cells into the pores of the porous scaffold. In some embodiments, the cells aggregate in the pores of the foam scaffold to form spheroids. The pores can be sized to confine at least some of the spheroids within the pores. As an aspect of embodiments, the cells do not adhere to the porous scaffold. In embodiments, the porous scaffold does not have a cell-adhesion coating. The method can further include harvesting at least one of the spheroids or a secreted material from the spheroids, where the secreted material can include an extracellular vesicle, proteins or other elements of the cell secretome. The method can include digesting any dissolvable foam scaffold by exposing the dissolvable foam scaffold to an enzyme and/or a chelating agent. In some embodiments, the perfusing of the cell culture medium includes continuously passing cell culture medium over the porous scaffold.

According to aspects of the present disclosure, a dissolvable foam scaffold for cell culture is provided herein. In some embodiments, the dissolvable foam scaffold includes an ionotropically crosslinked polygalacturonic acid compound selected from at least one of: pectic acid; partially esterified pectic acid, partially amidated pectic acid and salts thereof; and a water-soluble polymer having surface activity and having a hydrophilic-lipophilic balance (HLB) of greater than about 10, and preferably greater than about 20. Alternatively, the dissolvable foam scaffold may instead include an alginic acid as the ionotropically crosslinked polymer. In some embodiments, the HLB of the water-soluble polymer is greater than or equal to about 22. In some embodiments, the dissolvable foam scaffold does not contain glycerol, sorbitol, ethylene glycol, propylene glycol, polyethylene glycol. In some embodiments, the dissolvable foam scaffold includes glycerol, or one of sorbitol, ethylene glycol, propylene glycol and polyethylene glycol. According to aspects of embodiments, the dissolvable foam scaffold only includes a single water-soluble polymer having surface activity, and may not include any polymer having no surface activity. In some aspects of the present disclosure, a non-dissolvable foam scaffold for cell culture is provided herein. Cells may form spheroids in either the dissolvable foam scaffolds or the non-dissolvable foam scaffolds.

According to embodiments of the present disclosure, a method for forming a dissolvable foam scaffold is provided herein. The method includes forming a first aqueous mixture by adding an alginic acid or a polygalacturonic acid compound selected from at least one of: pectic acid; partially esterified pectic acid, partially amidated pectic acid and salts thereof to an aqueous solution; forming a second aqueous mixture by adding a water-soluble polymer having surface activity and a divalent metal salt to an aqueous solution; combining the first aqueous mixture with second first aqueous mixture to form a combined aqueous mixture; adding a gel inducing agent to the combined aqueous mixture to form a foaming solution; and introducing gas bubbles into the foaming solution to form a foam scaffold. The water-soluble polymer has a hydrophilic-lipophilic balance (HLB) of greater than about 10, or greater than about 20. In embodiments, the HLB of the water-soluble polymer is greater than or equal to about 22. In aspects of embodiments, forming the second aqueous mixture includes adding a water soluble plasticizer to the second aqueous mixture, and may not include adding a second polymer having no surface activity. According to additional aspects of embodiments, the method does not comprise adding an emulsifying agent to any of the first aqueous mixture, the second aqueous mixture, the combined aqueous mixture, and the foaming solution. According to embodiments, the method includes adding a first amount of the gel inducing agent to the aqueous mixture before foaming and adding a second amount of the gel inducing agent to the aqueous mixture during or after foaming. A ratio of the second amount of gel inducing agent to the first amount of gel inducing agent is greater than about 2, or about 7 or greater.

According to embodiments of the present disclosure, a method for culturing cells in a dissolvable foam scaffold is provided herein. The method includes seeding cells in a dissolvable foam scaffold such that cells enter pores of the dissolvable foam scaffold, the dissolvable scaffold comprising: an ionotropically crosslinked alginic acid and salts thereof, or an ionotropically crosslinked polygalacturonic acid compound selected from at least one of: pectic acid; partially esterified pectic acid, partially amidated pectic acid and salts thereof; and a water-soluble polymer having surface activity and having a hydrophilic-lipophilic balance (HLB) of greater than about 20; and contacting the dissolvable foam scaffold with cell culture medium. According to various aspects of embodiments, the dissolvable foam scaffold has 0 wt. % of a water soluble plasticizer, and does not include polymer having no surface activity. According to other aspects of embodiments, the dissolvable foam scaffold may contain a plasticizer, including a water soluble plasticizer.

According to aspects of the present disclosure, a method for producing spheroids or byproducts of spheroids is provided that comprises (a) providing a bioreactor comprising a cavity for culturing cells, an inlet and outlet to the cavity, and a porous scaffold, (b) inserting cells of a cell type into the porous scaffold to form spheroids, and (c) perfusing a cell culture medium through the cavity to culture the spheroids. The porous scaffold comprises pores and interconnects (passages) between the pores. In some embodiments, the step of inserting the cells of a cell type into the porous scaffold comprises seeding the cells into the porous scaffold. The cells substantially aggregate in the pores of the porous scaffold to form spheroids. In some embodiments, at least 80% of the cells in the pores of the porous scaffold aggregate to form spheroids. In some embodiments, the pores and passages between the pores are sized to confine at least some of the spheroids within the porous scaffold. In some embodiments, the method comprises the step of harvesting at least one of the spheroids or at least one byproduct of the spheroids. In some embodiments, the byproduct of the spheroids that is harvested is an extracellular vesicle. In some embodiments, the step of perfusing the cell culture medium comprises continuously passing cell culture medium over the porous scaffold.

In some embodiments, the porous scaffold is comprised of materials that are non-adherent to cells or the porous scaffold material is treated to be non-adherent to cells, or a combination thereof. In some embodiments, the porous scaffold is non-adherent to cells when perfused at a linear velocity from about 1.0×10⁻⁵ m/s to about 5.0×10⁻⁴ m/s (Darcy velocity). In some embodiments, the porous scaffold is non-adherent to at least 80% of the cells in the bioreactor.

The porous scaffold comprises pores, interconnects (passages) between pores that allow fluid to pass between pores, and a number of interconnects per pore. In some embodiments, at least 70% of the pores have a pore diameter from about 200 μm to about 1000 μm, when measured in a dried scaffold. In some embodiments, at least 80% of the pores have a pore diameter from about 400 μm to about 800 μm, when measured in a dried scaffold. In some embodiments, at least 70% of the passages have a maximum passage width from about 30 μm to about 500 μm, when measured in a dried scaffold. In some embodiments, at least 80% of the passages have a maximum passage width from about 60 μm to about 400 μm, when measured in a dried scaffold. In some embodiments, at least 70% of the pores have between 5 and 18 passages per pore. In some embodiments, at least 80% of the pores have from about 6 to about 14 passages per pore.

In some embodiments, the porous scaffold comprises an ionotropically crosslinked alginic acid or salts thereof, or an ionotropically crosslinked polygalacturonic acid compound selected from at least one of: pectic acid; partially esterified pectic acid, partially amidated pectic acid, and salts thereof. In some embodiments, the porous scaffold is dissolvable. In some embodiments with a dissolvable scaffold, the method also comprises the step of digesting the dissolvable scaffold by exposing it to an enzyme. In some embodiments, this exposing step may include exposing the scaffold to between about 1 U and about 200 U of the enzyme. In some embodiments, the enzyme for dissolving the scaffold comprises a non-proteolytic enzyme. In some embodiments, the nonproteolytic enzyme is selected from the group consisting of pectinolytic enzymes, pectinases, and alginate lyase. In some embodiments with a dissolvable scaffold, the method also comprises the step of exposing the dissolvable porous scaffold to a chelating agent. In some embodiments, this exposing step may include exposing the scaffold to between about 1 mM and about 200 mM of the chelating agent. In some embodiments, the digestion of the dissolvable porous scaffold is complete in less than about 1 hour.

According to aspects of the present disclosure, a bioreactor is provided that comprises a cavity for culturing cells, an inlet and outlet to the cavity, and a porous scaffold in the cavity. The porous scaffold comprises pores and passages between pores, is non-adherent to cells, and is configured to contain spheroids. In some embodiments, at least 75% of the pores have a pore diameter from about 200 μm to about 1000 μm, when measured in a dried scaffold. In some embodiments, at least 75% of the passages have a maximum passage width from about 30 μm to about 500 μm, when measured in a dried scaffold. In some embodiments, at least 75% of the pores have between 5 and 18 passages per pore. In some embodiments, the porous scaffold comprises an ionotropically crosslinked polysaccharide chosen from alginic acid and salts thereof, pectic acid and salts thereof, partly esterified pectic acid and salts thereof, partly amidated pectic acid and salts thereof, or a combination thereof. In one specific embodiment, the ionotropically crosslinked polysaccharide is polygalacturonic acid. In some embodiments, the porous scaffold of the bioreactor is dissolvable. In some embodiments, the porous scaffold of the bioreactor is a foam scaffold. In some embodiments, the porous scaffold is dissolved by pectinase or alginate lyase.

According to aspects of the present disclosure, a porous scaffold for culturing spheroids or byproducts from spheroids is provided that comprises pores, passages between the pores, and a number of passages per pore. This porous scaffold has at least 75% of the pores having a pore diameter from about 200 μm to about 1000 μm when measured in a dried scaffold, at least 75% of the passages having a maximum width of the passages from about 30 μm to about 500 μm when measured in a dried scaffold, and at least 75% of the pores have a number of passages between 5 and 18 passages per pore. This porous scaffold also is non-adherent to cells and is configured to grow spheroids in the pores, from cells of a cell type. In some embodiments of this porous scaffold, the cells of a cell type are chosen from a primary cell line and an immortalized cell line. In some embodiments, the primary cell line is bone-marrow derived human mesenchymal stem cells and the immortalized cell line is HEK293T cells.

According to aspects of the present disclosure, a perfusion bioreactor is provided that comprises a cavity for culturing cells, an inlet and outlet to the cavity, and a porous scaffold in the cavity. The porous scaffold of this perfusion bioreactor is non-adherent to cells and comprises pores and passages between the pores. This porous scaffold further has at least 75% of the pores having a pore diameter from about 200 μm to about 1000 μm when measured in a dried scaffold, at least 75% of the passages have a maximum width from about 30 μm to about 500 μm when measured in a dried scaffold, at least 75% of the pores have between 5 and 18 passages per pore, and the porous scaffold is adapted for growing spheroids or byproducts of spheroids. In some embodiments, the perfusion bioreactor is configured to retain at least 20% of any cells that are added through the inlet to the cavity. In some embodiments, the perfusion bioreactor is configured to retain at least 20% of any cells that are added through the inlet to the cavity. In some embodiments, the bioreactor is configured to retain at least 60% of any cells that are added through the inlet to the cavity. In some embodiments, the perfusion bioreactor is adapted for producing extracellular vesicles as the byproduct of spheroids. In some embodiments, the extracellular vesicles produced by the perfusion bioreactor are produced in an amount between about 1×10³ EVs per cell and about 1×10⁷ EVs per cell. In some embodiments, these extracellular vesicles are produced from perfusion with a linear velocity between about 1.0×10⁻⁵ m/s and about 5.0×10⁻⁴ m/s (Darcy velocity). In some embodiments, increasing the perfusion flow rate increases the number of extracellular vesicles produced per cell. In some embodiments, the number of extracellular vesicles produced per cell with the perfusion bioreactor is greater than the number of extracellular vesicles produced with a static 2D microwell plate.

In some embodiments, wound healing by the extracellular vesicles produced by the perfusion bioreactor is greater than wound healing by extracellular vesicles produced in a 2D flask for 3D cultures, at 6 hours post-injury as measured by a wound healing assay with HT-1080 cells. In some embodiments, wound healing with about 2×10⁹ extracellular vesicles produced by the perfusion bioreactor has at least 5% better wound closure than wound healing with 2×10⁹ extracellular vesicles produced in a 2D flask for 3D cultures, as measured by a wound healing assay with HT-1080 cells at 6 hours post-injury.

According to aspects of the present disclosure, a foamed scaffold product is provided herein. The foamed scaffold product is formed from a composition including an ionotropically crosslinked alginic acid and salts thereof, or an ionotropically crosslinked polygalacturonic acid compound selected from at least one of: pectic acid; partially esterified pectic acid, partially amidated pectic acid and salts thereof; at least one first water-soluble polymer having surface activity and having a hydrophilic-lipophilic balance (HLB) of greater than about 20; and 0 wt. % water soluble plasticizer. In other embodiments, the water soluble plasticizer may be present in values greater than 0 wt %.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary and are intended to provide an overview or framework to understanding the nature and character of the claims. The accompanying drawings are included to provide a further understanding and are incorporated in and constitute a part of this specification. The drawings illustrate one or more embodiment(s), and together with the description serve to explain principles and operation of the various embodiments.

### BRIEF DESCRIPTION OF THE DRAWINGS

The disclosure will be understood more clearly from the following description and from the accompanying figures, given purely by way of non-limiting example, in which:

FIG. 1 is a perspective view of a dissolvable foam scaffold in accordance with the present disclosure;

FIG. 2 shows a SEM image of a foam scaffold prepared in Example 1;

FIG. 3 shows a phase contrast image of spheroids formed from Vero cells in the pores of a foam scaffold prepared in Example 1, according to embodiments;

FIG. 4 shows a SEM image of a foam scaffold prepared in Example 3, according to embodiments;

FIG. 5A is an illustration of a foam scaffold for spheroid production in a culture plate, according to embodiments;

FIG. 5B is an illustration of a foam scaffold for spheroid or cell product production in a perfusion bioreactor, according to embodiments;

FIG. 6A is a photograph of an uncoated foam scaffold made from composition XP49, according to embodiments;

FIG. 6B is a photograph of an uncoated foam scaffold made from composition XP64, according to embodiments;

FIG. 6C is a photograph of an uncoated foam scaffold made from composition XP76, according to embodiments;

FIG. 6D is a photograph of an uncoated foam scaffold made from composition FMXP005, according to embodiments;

FIG. 6E is a plot representing measured scaffold pore size in different scaffolds, according to embodiments;

FIG. 6F is a plot representing measured scaffold interconnect size in different scaffolds, according to embodiments;

FIG. 6G is a plot representing the number of interconnects per pore in different scaffolds, according to embodiments;

FIG. 7A is a fluorescent microscopy image of HEK293T spheroids 1 day and 5 days after seeding the scaffold with HEK293 cells, according to embodiments. The left column depicts spheroids in a foam scaffold with the XP64 composition. The right column depicts spheroids in a foam scaffold with the XP76 composition.

FIG. 7B is a fluorescent microscopy image of hMSC spheroids 1 day and 7 days after seeding the scaffold with hMSC cells, according to embodiments. The left column depicts spheroids in a foam scaffold with the XP64 composition. The right column depicts spheroids in a foam scaffold with the XP76 composition.

FIG. 8 is a bar graph showing the increase in HEK293T at 1 day and 5 days after seeding the cells in FIG. 7A, and the increase in hMSC cells at 1 day and 7 days after seeding the cells in FIG. 7B, according to embodiments;

FIG. 9A is a graph of cell seeding data in a perfusion bioreactor, according to embodiments;

FIG. 9B is a bar graph showing the percentage of recovered cells from a scaffold 1 day after seeding, according to embodiments;

FIG. 10 shows fluorescent microscopy images from two perfusion bioreactors one day after HEK293T cells were seeded into foam scaffolds, according to embodiments;

FIG. 11 shows fluorescent microscopy images of different foam scaffold composition and the spheroids after 48 hours of perfusion at 1 mL/min and 10 mL/min perfusion flow rates, according to embodiments;

FIG. 12 is a bar graph showing cell count of cells recovered from the scaffolds in FIG. 11 at 48 hours after beginning of perfusion, according to embodiments;

FIG. 13A is a fluorescent microscopy image of hMSC spheroids in a foam scaffold after 48 hours of perfusion at 10 mL/min, according to embodiments;

FIG. 13B is a fluorescent microscopy image of hMSC spheroids in a foam scaffold after 48 hours of perfusion at 2 mL/min, according to embodiments;

FIG. 13C is a brightfield contrast phase microscopy image of 2D adherent hMSCs in on a 2D static surface after 48 hours, according to embodiments;

FIG. 14A is a graph of the size distribution of EVs/particles produced by hMSCs in a 3D foam scaffold using a perfusion flow rate of 10 mL/min based on a MADLS analysis, according to embodiments;

FIG. 14B is a graph of the size distribution of EVs/particles produced by hMSCs in a 3D foam scaffold using a perfusion flow rate of 2 mL/min based on a MADLS analysis, according to embodiments;

FIG. 14C is a graph of the size distribution of EVs/particles produced by hMSCs in a 2D static environment based on a MADLS analysis, according to embodiments;

FIG. 15A is a plot of the concentration of EVs/particles produced by hMSCs in a 3D foam scaffold using a perfusion flow rate of 10 mL/min based on a MADLS analysis, according to embodiments;

FIG. 15B is a plot of the concentration of EVs/particles produced by hMSCs in a 3D foam scaffold using a perfusion flow rate of 2 mL/min based on a MADLS analysis, according to embodiments;

FIG. 15C is a plot of the concentration of EVs/particles produced by hMSCs in a 2D static environment based on a MADLS analysis, according to embodiments;

FIG. 16 is a bar graph of the number of EVs per million cells for each of the three culture conditions from FIGS. 15A-15C based on a MADLS analysis, according to embodiments;

FIG. 17 is a bar graph of the number of EVs per millions cells for each of the three culture conditions based on an ELISA against CD63 positive EVs analysis, according to embodiments;

FIG. 18 is a picture of a Western blot analysis against CD81 and TSG101 on EV isolated samples produced according to a 10 ml/min perfusion 3D foam culture and a static 2D culture, according to embodiments;

FIG. 19 shows brightfield contrast phase microscopy images of the HT1080 wound healing assay at 0 and 8 hours after performing the wound and treating the cells with 2×10⁹ CD63+EVs or no treatment, according to embodiments;

FIG. 20 is a plot of EVs produced by hMSCs in different conditions using analysis by in vitro would healing assay, according to embodiments;

FIG. 21A shows a fluorescent microscopy image of hMSC spheroids observed after 48 hours of exposure to EV collection media through perfusion at 10 mL/min, according to embodiments. The spheroids were stained with calcein AM before the photographs;

FIG. 21B shows a brightfield contrast phase microscopy image of hMSC spheroids in a microcavity vessel observed after 48 hours of exposure to EV collection media with agitation at 35 rpm, according to embodiments;

FIG. 21C shows a brightfield contrast phase microscopy image of 2D adherent hMSC in a T-75 CellBIND® flask observed after 48 hours of exposure to EV collection media with static conditions, according to embodiments;

FIG. 22A is a distribution graph of particle sizes by MADLS analysis of EVs produced by hMSC spheroids after 48 hours of exposure to EV collection media through perfusion at 10 mL/min, according to embodiments;

FIG. 22B is a distribution graph of particle sizes by MADLS analysis of EVs produced by hMSC spheroids after 48 hours of exposure to EV collection media with agitation at 35 rpm, according to embodiments;

FIG. 22C is a distribution graph of particle sizes by MADLS analysis of EVs produced by 2D adherent hMSC after 48 hours of exposure to EV collection media with static conditions, according to embodiments;

FIG. 22D is a bar graph of the amount of particles and EVs produced 48 hours of exposure to EV collection media, according to embodiments;

FIG. 23A is a bar graph of the number of EVs produced by hMSC spheroids and 2D adherent hMSC in different conditions after 48 hours of exposure to EV collection media, according to embodiments, as analyzed by ELISA against CD63;

FIG. 23B shows Western blots using CD81 and TSG101 markers of EVs produced by hMSC spheroids and 2D adherent hMSC in different conditions after 48 hours of exposure to EV collection media, according to embodiments;

FIG. 24 is a plot of the results of an EV functional assay using a wound healing assay with HT-1080 cells using a control and EVs produced by hMSC in different conditions after 48 hours of exposure to EV collection media, according to embodiments;

FIG. 25 shows fluorescent microscopy images of hMSCs after 48 hours of exposure to EV collection media through perfusion at 20, 10, 2 and 0.5 mL/min and 2D adherent hMSCs, according to embodiments. The spheroids were stained with calcein AM before the photographs;

FIG. 26A shows plots of the average diameters of EVs that were obtained in clarified EV conditioned perfusion media at the indicated conditions, using MADLS analysis, according to embodiments. The EVs were produced by hMSCs from the bioreactor;

FIG. 26B shows plots of the total EVs that were obtained in clarified EV conditioned perfusion media at the indicated conditions, using MADLS analysis, according to embodiments. The EVs were produced by hMSCs from the bioreactor;

FIG. 27A shows distribution graphs of the diameters of EVs that were obtained from purified EV samples at the indicated conditions, using MADLS, according to embodiments. The EVs were produced by hMSCs from the bioreactor;

FIG. 27B shows a bar graph of the total EVs that were obtained from purified EV samples at the indicated conditions, using MADLS, according to embodiments. The EVs were produced by hMSCs from the bioreactor;

FIG. 27C shows a bar graph of EVs produced per million of hMSC cells calculated from the total EVs in the purified EV samples and the total number of cells in the bioreactor, according to embodiments;

FIG. 28A shows a bar graph of the total numbers of EVs that were obtained from purified EV samples at the indicated conditions, using ELISA CD63 analysis, according to embodiments;

FIG. 28B shows a bar graph of EVs produced per million of hMSC cells calculated from the total EVs in the purified EV samples and the total number of cells in the bioreactor, according to embodiments;

FIG. 29 shows a bar graph of the wound healing % level by EVs obtained from purified EV conditioned perfusion media at the indicated conditions as observed at 6 hours after injury using an HT-1080 wound healing assay, according to embodiments.

### DETAILED DESCRIPTION

Reference will now be made in detail to the present embodiment(s), an example(s) of which is/are illustrated in the accompanying drawings. Whenever possible, the same reference numerals will be used throughout the drawings to refer to the same or like parts.

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

Unless otherwise stated, the use of individual numerical values is stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

As used herein, “have,” “having,” “include,” “including,” “comprise,” “comprising” or the like are used in their open ended sense, and generally mean “including, but not limited to.”

“Optional” or “optionally” means that the subsequently described element, component or circumstance may or may not occur, so that the description includes instances where the element, component, or circumstance occurs and instances where it does not.

As used herein, “shear stress” or “wall shear stress” are interchangeable, and refers to the tangential force per unit area that is exerted by the flow of fluid through the pores of the 3D scaffold on the spheroids cultured inside these pores.

All scientific and technical terms used herein have meanings commonly used in the art unless otherwise specified. The definitions provided herein are to facilitate understanding of certain terms used frequently herein and are not meant to limit the scope of the present disclosure.

The present disclosure is described below, at first generally, then in detail on the basis of several exemplary embodiments. The features shown in combination with one another in the individual exemplary embodiments do not all have to be realized. In particular, individual features may also be omitted or combined in some other way with other features shown of the same exemplary embodiment or else of other exemplary embodiments.

Embodiments of the present disclosure relate to methods for producing large-scale amounts of spheroids from isolated cells and for producing high concentrations of extracellular vesicles and other cellular byproducts secreted from these spheroids. Embodiments herein provide, for the first time, methods to produce spheroids through cell suspension perfusion inside a bioreactor with a scaffold, as well as methods to produce EVs and other secreted byproducts from cells in spheroids under perfusion inside a bioreactor. Embodiments include forming spheroids in dissolvable foam scaffolds, after which the spheroids can either be recovered from the scaffold and used for other applications or used directly inside the scaffold for specific application like EV production. In other embodiments, a non-dissolvable scaffold enables spheroid formation in the non-dissolvable scaffold, after which the spheroids can be used directly inside the scaffold for specific application like EV production. According to embodiments, the dissolvable and non-dissolvable foam scaffolds of the present disclosure for growing and cultivating spheroids can be used without an external cell-adhesion coating on the scaffold, thus preventing cell adhesion to the scaffold. After seeding cells in the scaffold, the cells will form spheroids inside the scaffold's pores. Depending on the cell type and pore size, these spheroids have the capacity to grow and, at least with dissolvable scaffolds, the spheroids can be recovered after formation and used for other applications, including, for example, toxicity assays or other applications that would be appreciated by a person of ordinary skill in the art.

Existing methods used to generate spheroids generally involve growing the spheroids in plates with multiple cavities treated to prevent cell adhesion. The limited number of cavities on these plates leads to a limited number of spheroids. In addition, the large footprint of such plates leads to an inefficient use of space compared to 3D scaffolds disclosed herein. The high density of pores inside the scaffold leads to a high density of spheroids that is much higher than any current method used to generate spheroids. Other methods to generate spheroids do not allow an easy recovery of these spheroids, especially in large quantities.

Cells cultured in spheroids can produce useful byproducts (the secretome) into the extracellular space around the cells. The secretome is the set of proteins and other biological elements expressed by a cell and secreted into the extracellular space. One element of the secretome are EVs. According to embodiments of this disclosure, methods are provided that allow the large-scale production of functional EVs by generating spheroids inside a porous material and exposing these spheroids to media perfusion in a bioreactor.

Advantages of embodiments of this disclosure include large-scale spheroid formation, which can be recovered if a dissolvable scaffold is used. For spheroids whose growth is partially dependent on the pore size, controlling the spheroid size via the engineered pore size of the scaffold material, may help provide a homogenous population of spheroids, which is important for some downstream applications. In addition, aspects of embodiments herein provide the advantage of large-scale EV production and other cell secretome or particle productions (e.g., exosomes) from spheroids under perfusion in the scaffold. The amount of EVs, particles, and/or secretome production by the spheroids can also be controlled through the dependence of these quantities on the perfusion flow rate. Further, embodiments of this disclosure are scalable to enable production of spheroids, EVs, and other cell byproducts at various scales, including large scale production. For example, by increasing the diameter of the scaffold, spheroid and EV/secretome productions are increased.

Embodiments of the present disclosure relate to dissolvable and non-dissolvable foam scaffolds for cell culture and methods of making dissolvable foam scaffolds. Embodiments of the present disclosure further relate to methods of cell culture of adherent cells, cell aggregates, or spheroids, in dissolvable and non-dissolvable foam scaffolds. Furthermore, embodiments of the present disclosure relate to bioreactors and bioreactors systems including dissolvable and non-dissolvable foam scaffolds. As will become clearer in the discussions below, in some embodiments, the foam scaffolds as disclosed herein are described as being dissolvable and insoluble. As used herein, the term “insoluble” is used to refer to a material or combination of materials that is not soluble, and that remains crosslinked, under conventional cell culture conditions which include, for example, cell culture media. Also as used herein, the term “dissolvable” is used to refer to a material or combination of materials that is digested when exposed to an appropriate concentration of an enzyme that digests or breakdowns the material or combination of materials. The dissolvable and non-dissolvable foam scaffolds described herein are porous scaffolds having an open pore architecture and highly interconnected pores. The pores of the scaffolds provide a protected environment for the culturing of cells where the cell-to-cell interactions and formation of extracellular matrix in a 3D fashion are aided. The dissolvable foam scaffolds may be completely digested which allows for harvesting cells without damaging the cells using protease treatment and/or mechanical harvesting techniques.

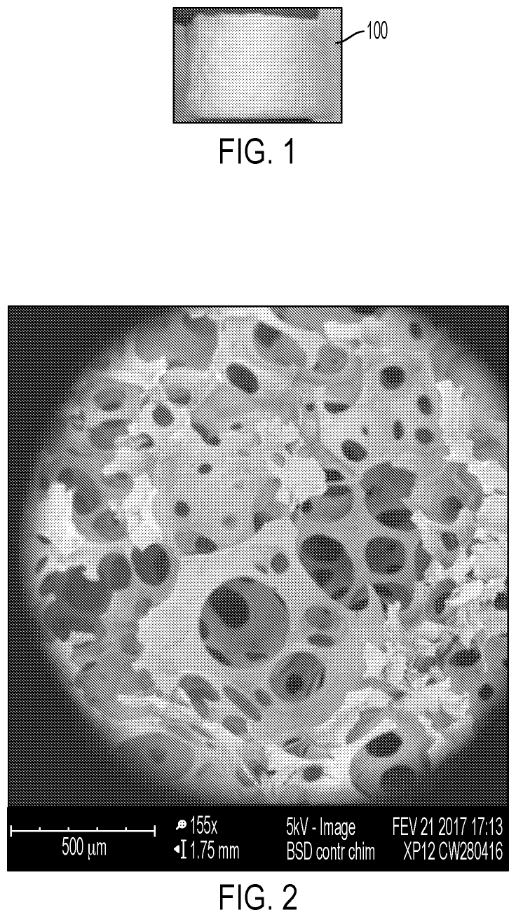

FIG. 1 is a perspective view of a dissolvable foam (porous) scaffold 10 in accordance with the present disclosure. As will be described in further detail below and as will become more clear from the other figures of the present disclosure, dissolvable foam scaffold 10 is a porous foam that includes an open pore architecture. Dissolvable foam scaffold 10 has a porosity of from about 85% to about 96% and an average pore size diameter of between about 50 μm and about 500 μm. Dissolvable foam scaffold 10 provides a protected environment within the pores of the foam scaffold for the culturing of the spheroids. Additionally, dissolvable foam scaffold 10 is also dissolvable when exposed to an appropriate enzyme that digests or breakdowns the material which facilitates harvesting of the spheroids cultured in the scaffold without damaging the cells.

Dissolvable foam scaffolds as described herein include at least one ionotropically crosslinked polysaccharide and can be used for culturing three-dimensional cell cultures (for example, spheroids or organoids) and byproducts of these cultures (for example, extracellular vesicles and exosomes). Generally, polysaccharides possess attributes beneficial to cell culture applications. Polysaccharides are hydrophilic, non-cytotoxic and stable in culture medium. Examples include pectic acid, also known as polygalacturonic acid (PGA), or salts thereof, partly esterified pectic acid or salts thereof, or partly amidated pectic acid or salts thereof. Another example of an ionotropically crosslinked polysaccharide that can be used for the scaffolds of the present disclosure includes alginic acid or salts thereof. Pectic acid can be formed via hydrolysis of certain pectin esters. Pectins are cell wall polysaccharides and in nature have a structural role in plants. Major sources of pectin include citrus peel (e.g., peels from lemons and limes) and apple peel. Pectins are predominantly linear polymers based on a 1,4-linked alpha-D-galacturonate backbone, interrupted randomly by 1,2-linked L-rhamnose. The average molecular weight ranges from about 50,000 to about 200,000 Daltons.

The polygalacturonic acid chain of pectin may be partly esterified, e.g., methyl groups and the free acid groups may be partly or fully neutralized with monovalent ions such as sodium, potassium, or ammonium ions. Polygalacturonic acids partly esterified with methanol are called pectinic acids, and salts thereof are called pectinates. The degree of methylation (DM) for high methoxyl (HM) pectins can be, for example, from 60 to 75 mol % and those for low methoxyl (LM) pectins can be from 1 to 40 mol %. The degree of esterification of partly esterified polygalacturonic acids as described herein may be less than about 70 mol %, or less than about 60 mol %, or less than 50 mol %, or even less than about 40 mol %, and all values therebetween. Without wishing to be bound by any particular theory, it is believed that a minimum amount of free carboxylic acid groups (not esterified) facilitates a degree of ionotropic crosslinking which allow for the formation of a dissolvable scaffold which is insoluble.

Alternatively, the polygalacturonic acid chain of pectin may be partly amidated. Polygalacturonic acids partly amidated pectin may be produced, for example, by treatment with ammonia. Amidated pectin contains carboxyl groups (~COOH), methyl ester groups (~COOCH₃), and amidated groups (—CONH₂). The degree of amidation may vary and may be, for example, from about 10% to about 40% amidated.

According to embodiments of the present disclosure, dissolvable foam scaffolds as described herein may include a mixture of pectic acid and partly esterified pectic acid. Blends with compatible polymers may also be used. For example, pectic acid and/or partly esterified pectic acid may be mixed with other polysaccharides such as dextran, substituted cellulose derivatives, alginic acid, starches, glycogen, arabinoxylans, agarose, etc. Glycosaminoglycans like hyaluronic acid and chondroitin sulfate, or various proteins such as elastin, fibrin, silk fibroin, collagen and their derivatives can be also used. Water soluble synthetic polymers can be also blended with pectic acid and/or partly esterified pectic acid. Exemplary water soluble synthetic polymers include, but are not limited to, polyalkylene glycol, poly(hydroxyalkyl (meth)acrylates), poly(meth)acrylamide and derivatives, poly(N-vinyl-2-pyrrolidone), and polyvinyl alcohol.

According to embodiments of the present disclosure, dissolvable foam scaffolds as described herein may further include at least one first polymer. The at least one first polymer is water soluble, non-ionotropically crosslinkable and has surface activity. As used herein, the term “surface activity” refers to the activity of an agent to lower or eliminate the surface tension (or interfacial tension) between two liquids or between a liquid and a solid or between gas and liquid. The at least one first polymer may have a hydrophilic-lipophilic balance (HLB) of greater than about 8 or even greater than about 10. For example, the at least one first polymer may have an HLB of between about 8 and about 40 or between about 10 and about 40. The at least one first polymer may have an HLB of between about 8 and about 15, or even between about 10 and about 12. HLB provides a reference for the lipophilic or hydrophilic degree of a polymer. A larger HLB value indicates stronger hydrophilicity, while a smaller HLB value indicates a stronger lipophilicity. In general, the HLB value varies in the range of from 1 to 40 and the hydrophilic-lipophilic transition is often considered to be between about 8 and about 10. When the HLB value is less than the hydrophilic-lipophilic transition, the material is lipophilic, and when the HLB value is greater than the hydrophilic-lipophilic transition the material is hydrophilic. In some embodiments, the HLB value of the at least one first polymer is 10 or greater. In some embodiments, the HLB value of the at least one first polymer is 15 or greater. In some embodiments, the HLB value of the at least one first polymer is 20 or greater. In yet other embodiments, the HLB value of the at least one first polymer is between 10 and 40, between 15 and 40, between 20 and 40, or between 20 and 30.

Exemplary first polymers in accordance with embodiments of the present disclosure may be any of cellulose derivatives, proteins, synthetic amphiphilic polymers, and combinations thereof. Exemplary cellulose derivatives include, but are not limited to, hydroxyethylcellulose (HEC), hydroxypropylcellulose (HPC), methylcellulose (MC), hydroxyethylmethylcellulose (HEMC), and hydroxypropyl-methylcellulose (HPMC). Exemplary proteins include, but are not limited to, bovine serum albumin (BSA), gelatine, casein and hydrophobins. Exemplary synthetic amphiphilic polymers include, but are not limited to, a poloxamer available under the trade name Synperonics® (commercially available from Croda International, Snaith, United Kingdom), a poloxamer available under the trade name Pluronics® (commercially available from BASF Corp., Parsippany, NJ) and a poloxamer available under the trade name Kolliphor® (commercially available from BASF Corp., Parsippany, NJ).

Dissolvable foam scaffolds as described herein may further include at least one second polymer. The at least one second polymer is water soluble and has no surface activity. Exemplary second polymers may be any of synthetic polymers, semisynthetic polymers, natural polymers and combinations thereof. Exemplary synthetic polymers include, but are not limited to, polyethylene glycol, polyvinyl pyrrolidone, polyvinyl alcohol, carboxyvinyl polymer, polyacrylic acid, polyacrylamide, homopolymer and copolymer of N-(2-Hydroxypropyl) methacrylamide, polyvinyl methyl ether-maleic anhydride, and polyethylene oxide/polypropylene oxide block copolymers. Exemplary semisynthetic polymers include, but are not limited to, dextran derivatives, carboxymethyl cellulose, hydroxyethyl cellulose and derivatives, methylcellulose and derivatives, ethylcellulose cellulose, ethyl hydroxyethyl cellulose, and hydroxypropyl cellulose. Exemplary natural polymers include, but are not limited to, starch and starch derivatives, polymers obtained by microbial fermentation such as curdlan, pullulan and gellan gum, xanthan gum, dextran, proteins such as albumin, casein and caseinates, gelatin, seaweed extracts such as agar, alginates and carrageenan, seed extracts such as guar gum and derivatives and locust bean gum, hyaluronic acid, and chondroitin sulfate.

Dissolvable foam scaffolds as described herein may be crosslinked to increase their mechanical strength and to prevent the dissolution of the scaffolds when placed in contact with cell culture medium. Crosslinking may be performed by ionotropic gelation as described below wherein ionotropic gelation is based on the ability of polyelectrolytes to crosslink in the presence of multivalent counter ions to form crosslinked scaffolds. Without wishing to be bound by any particular theory, it is believed that ionotropic gelation of the polysaccharide of the dissolvable foam scaffolds is the result of strong interactions between divalent cations and the polysaccharide.

It should be understood that scaffolds for forming and producing spheroids and the cell byproducts of the present disclosure are dissolvable scaffolds in only some embodiments. Non-dissolvable 3D scaffolds with similar pore size, interconnect (passage) size, number of interconnects per pore as any of the dissolvable 3D scaffolds also described herein will work to form spheroids and cell byproducts. Such non-dissolvable scaffolds may be made from, for example, polytetrafluoroethylene (PTFE), polyethylene terephthalate (PET), polystyrene (PS), polypropylene (PP), polyethylene (PE), polyester (PE), polyamide (PA), polyvinylidene fluoride (PVDF), acrylamides, and agar, or a combination thereof. In embodiments where the non-dissolvable scaffold material is cell adherent, the non-dissolvable scaffold material is coated with material that is non-adherent or is otherwise made non-adherent by chemical modifications or other known method of making the scaffold material non-adherent.

According to embodiments of the present disclosure, the dissolvable and non-dissolvable scaffolds as described herein are porous foam scaffolds. Foam scaffolds as described herein may have a porosity of from about 85% to about 96%. For example, foam scaffolds as described herein may have a porosity of from about 91% to about 95%, or about 94% to about 96%. As used herein, the term “porosity” refers to the measure of open pore volume in the dissolvable scaffold and is referred to in terms of % porosity, wherein % porosity is the percent of voids in the total volume of the dissolvable foam scaffold.

Foam scaffolds as described herein may have an average pore size diameter of between about 200 μm and about 1000 μm, when measured in a dried scaffold. Drying of foam (porous) scaffolds can be performed by any method used by those of ordinary skill in the art, but preferably, by the method of freeze-drying. The diameter of the pore is the widest distance across the pore and is measured by scanning electron microscopy (“SEM”). In some embodiments, average pore size diameter in a dried scaffold may be between about 250 μm and about 650 μm, or between about 300 μm and about 600 μm, or even between about 350 μm and about 500 μm, and all values therebetween. In some embodiments, the average pore size diameter may be between about 450 μm and about 850 μm, between about 500 μm and about 700 μm, or between about 550 μm and about 600 μm, when measured in a dried scaffold. In some embodiments, the average pore size diameter may be between about 300 μm and about 650 μm, between about 350 μm and about 550 μm, or between about 400 μm and about 525 μm, when measured in a dried scaffold. In some embodiments, the average pore size diameter may be between about 350 μm and about 1000 μm, between about 450 μm and about 850 μm, or between about 550 μm and about 650 μm, when measured in a dried scaffold. The average pore size diameter is the average of the pore diameters of the pores in the scaffold.

In some embodiments, at least 50% of the pores have a pore diameter from about 200 μm to about 1000 μm, when measured in a dried scaffold. In another embodiment, at least 50%, 55%, 60%, 65%, 70%, 75%, 80%, 85%, 90%, 95%, or more (or any value between 50% and 100%) of the pores have a pore diameter from about 200 μm to about 1000 μm, when measured in a dried scaffold. In one specific embodiment, between 65% and 85% of the pores have a pore diameter from about 200 μm to about 1000 μm, when measured in a dried scaffold. In another specific embodiments, at least 70% of the pores have a pore diameter from about 200 μm to about 1000 μm, when measured in a dried scaffold. In some embodiments, at least 50% of the pores have a pore diameter from about 400 μm to about 800 μm, when measured in a dried scaffold. In another embodiment, at least 50%, 55%, 60%, 65%, 70%, 75%, 80%, 85%, 90%, 95%, or more (or any value between 50% and 100%) of the pores have a pore diameter from about 400 μm to about 800 μm, when measured in a dried scaffold. In one specific embodiment, between 70% and 90% of the pores have a pore diameter from about 400 μm to about 800 μm, when measured in a dried scaffold. In another specific embodiment, at least 80% of the pores have a pore diameter from about 400 μm to about 800 μm, when measured in a dried scaffold.

Scaffolds as described herein may have a wet density of less than about 0.40 g/cc. For example, scaffolds as described herein may have a wet density of less than about 0.35 g/cc, or less than about 0.30 g/cc, or less than about 0.25 g/cc. Scaffolds as described herein may have a wet density of between about 0.16 g/cc and about 0.40 g/cc, or between about 0.16 g/cc and about 0.35 g/cc, or between about 0.16 g/cc and about 0.30 g/cc, or even between about 0.16 g/cc and about 0.25 g/cc, and all values therebetween. Scaffolds as described herein may have a dry density of less than about 0.20 g/cc. For example, scaffolds as described herein may have a dry density of less than about 0.15 g/cc, or less than about 0.10 g/cc, or less than about 0.05 g/cc. Scaffolds as described herein may have a dry density of between about 0.02 g/cc and about 0.20 g/cc, or between about 0.02 g/cc and about 0.15 g/cc, or between about 0.02 g/cc and about 0.10 g/cc, or even between about 0.02 g/cc and about 0.05 g/cc, and all values therebetween.

Several pore types are possible in foam (porous) scaffolds. Open pores allow for cellular access on both sides of the scaffold and allow for liquid flow and transport of nutrients through the dissolvable scaffold. Partially open pores allow for cellular access on one side of the scaffold, but mass transport of nutrients and waste products is limited to diffusion. Closed pores have no openings and are not accessible by cells or by mass transport of nutrients and waste products. Both dissolvable and non-dissolvable foam scaffolds as described herein have an open pore architecture and highly interconnected pores. Generally, the open pore architecture and highly interconnected pores enable migration of cells into the pores of the dissolvable foam scaffolds and also facilitate enhanced mass transport of nutrients, oxygen and waste products. The open pore architecture also influences cell adhesion and cell migration by providing a high surface area for cell-to-cell interactions and space for extracellular matrix (“ECM”) regeneration.

The porous scaffolds (dissolvable and non-dissolvable) of the present disclosure have passages (interconnects) between pores that allows fluid to flow through the scaffold. In some embodiments, at least 50% of the interconnects (passages) have a maximum passage width from about 30 μm to about 500 μm, when measured in a dried scaffold. As used herein, the measurements for passage width are values measured in a dried scaffold. In another embodiment, at least 50%, 55%, 60%, 65%, 70%, 75%, 80%, 85%, 90%, 95%, or more (or any value between 50% and 100%) of the interconnects (passages) have a maximum passage width from about 30 μm to about 500 μm, when measured in a dried scaffold. In one specific embodiment, between 65% and 85% of the passages have a maximum passage width from about 30 μm to about 500 μm, when measured in a dried scaffold. In another specific embodiment, at least 70% of the passages have a maximum passage width from about 30 μm to about 500 μm, when measured in a dried scaffold. In some embodiments, at least 50% of the passages have a maximum passage width from about 60 μm to about 400 μm, when measured in a dried scaffold. In another embodiment, at least 50%, 55%, 60%, 65%, 70%, 75%, 80%, 85%, 90%, 95%, or more (or any value between 50% and 100%) of the passages have a maximum passage width from about 60 μm to about 400 μm, when measured in a dried scaffold. In one specific embodiments, between 70% and 90% of the passages have a maximum passage width from about 60 μm to about 400 μm, when measured in a dried scaffold. In another specific embodiment, at least 80% of the passages have a maximum passage width from about 60 μm to about 400 μm, when measured in a dried scaffold. The maximum passage width is the widest part of a cross-sectional area across a passage of a dried scaffold, measured by scanning electron microscopy.

In some embodiments, the average interconnect diameter (i.e., the average maximum distance across a cross-sectional area of a passage space that links pores together) is between about 150 μm and about 500 μm, between about 250 μm and about 400 μm, or between about 275 μm and about 375 μm, when measured in a dried scaffold. In some embodiments, the average interconnect diameter is between about 30 μm and about 300 μm, between about 80 μm and about 250 μm, or between about 130 μm and about 200 μm, when measured in a dried scaffold. In some embodiments, the average interconnect diameter is between about 70 μm and about 400 μm, between about 120 μm and about 350 μm, or between about 170 μm and about 300 μm, when measured in a dried scaffold.

The porous scaffolds (dissolvable and non-dissolvable) of the present disclosure also have a number of interconnects (passages) that are present per pore. This number can generally range from 1 interconnect per pore to 40 interconnects per pore, or more. In some embodiments, at least 50% of the pores have between 5 and 18 passages per pore. In another embodiment, at least 50%, 55%, 60%, 65%, 70%, 75%, 80%, 85%, 90%, 95%, or more (or any value between 50% and 100%) of the pores have between 5 and 18 passages per pore. In one specific embodiment, between 65% and 85% of the pores have between 5 and 18 passages per pore. In another specific embodiment, at least 70% of the pores have between 5 and 18 passages per pore. In some embodiments, at least 50% of the pores have a number of passages per pore from about 6 to about 14 passages per pore. In another embodiment, at least 50%, 55%, 60%, 65%, 70%, 75%, 80%, 85%, 90%, 95%, or more (or any value between 50% and 100%) of the pores have a number of passages per pore from about 6 to about 14 passages per pore. In one specific embodiments, between 70% and 90% of the pores have a number of passages per pore from about 6 to about 14 passages per pore. In another specific embodiment, at least 80% of the pores have a number of passages per pore from about 6 to about 14 passages per pore. The number of interconnects (passages) per pore can be measured with scanning electron microscopy.

In some embodiments, the average number of interconnects (passages) per pore is between 6 and 20, between 8 and 18, or between 10 and 14. In some embodiments, the average number of interconnects (passages) per pore is between 4 and 16, between 6 and 16, or between 7 and 11. In some embodiments, the average number of interconnects (passages) per pore is between 6 and 12, or between 7 and 10. In some embodiments, the average number of interconnects (passages) per pore is between 12 and 18, between 13 and 17, or between 14 and 16.

For embodiments with dissolvable foam scaffolds, those scaffolds can be dissolved under certain circumstances. Dissolvable foam scaffolds as described herein are digested when exposed to an appropriate enzyme that digests or breakdowns the material. Non-proteolytic enzymes suitable for digesting the foam scaffolds, harvesting cells, or both, include pectinolytic enzymes or pectinases, which are a heterogeneous group of related enzymes that hydrolyze the pectic substances. Pectinases (polygalacturonase) are enzymes that break down complex pectin molecules to shorter molecules of galacturonic acid. Commercially available sources of pectinases are generally multi-enzymatic, such as Pectinex™ ULTRA SP-L (commercially available from Novozyme North American, Inc., Franklinton, NC), a pectolytic enzyme preparation produced from a selected strain of Aspergillus aculeatus. Pectinex™ ULTRA SP-L contains mainly polygalacturonase, (EC 3.2.1.15) pectintranseliminase (EC 4.2.2.2) and pectinesterase (EC 3.1.1.11). The EC designation is the Enzyme Commission classification scheme for enzymes based on the chemical reactions the enzymes catalyze. Non-proteolytic enzymes suitable for digesting foam scaffolds, harvesting cells, or both, also include alginate lyase (EC 4.2.2.3). Alginate lyase is an enzyme that breaks down alginic acid into shorter molecules. Commercially available sources of alginate lyase can be used, such as those from Sigma Alrich®.

According to some embodiments of the present disclosure, digestion of the dissolvable foam scaffolds also includes exposing the scaffold to a divalent cation chelating agent. Exemplary chelating agents include, but are not limited to, ethylenediaminetetraacetic acid (EDTA), cyclohexanediaminetetraacetic (CDTA), ethylene glycol tetraacetic acid (EGTA), citric acid and tartaric acid.

The time to complete digestion of dissolvable foam scaffolds as described herein may be less than about 1 hour. For example, the time to complete digestion of foam scaffolds may be less than about 45 minutes, or less than about 30 minutes, or less than about 15 minutes, or less than 5 minutes, or less than 1 minute, or between about 1 minute and about 25 minutes, or between about 3 minutes and about 20 minutes, or even between about 5 minutes and about 15 minutes.

According to embodiments of the present disclosure, methods for forming dissolvable foam scaffolds as described herein are also disclosed. Methods as described herein may include forming a first aqueous mixture which includes dissolving a polysaccharide in an aqueous solution. Polysaccharides may be those as described above, such as pectic acid or salts thereof, partly esterified pectic acid or salts thereof, or partly amidated pectic acid or salts thereof, and blends of such polysaccharides.

Methods for forming dissolvable foam scaffolds as described herein may further include forming a second aqueous mixture including a water insoluble divalent metal salt in an aqueous solution. Metals of the divalent metal salts may include, but are not limited to, magnesium, calcium, zinc, strontium, barium, and like cations, and combinations thereof. Anions of the divalent metal salts may include, but are not limited to, oxalates, tartrates, phosphates, carbonates, citrates, and like organic and inorganic anions, and combinations thereof.

According to embodiments of the present disclosure, forming a second aqueous mixture may further include adding the at least one first polymer as described above to the second aqueous mixture. Optionally, methods as described herein may further include adding the at least one second polymer as described above to the second aqueous mixture. According to embodiments of the present disclosure, the at least one first polymer and the at least one second polymer may be added to the second aqueous mixture separately or may be added to the second aqueous mixture together. When added as a mixture, the mixture may include about 50% of the at least one first polymer and about 50% of the at least one second polymer. For example, the mixture may include between about 35% and about 65% (and all values therebetween) of the at least one first polymer and between about 35% and about 65% (and all values therebetween) of the at least one second polymer.

According to embodiments of the present disclosure, forming a second aqueous mixture may further include adding at least one water soluble plasticizer to the second aqueous mixture. Plasticizers as described herein are non-toxic and do not affect the solubility of the polysaccharides of the dissolvable foam scaffolds. A plasticizer provides flexibility and softness to the resulting foam such that the resulting foam is soft and pliable. Plasticizers as described herein may include, but are not limited to, polyhydric alcohols such as glycerol, sorbitol, ethylene glycol, propylene glycol, polyethylene glycol and combinations thereof. Adding a water soluble plasticizer to the second aqueous mixture may include adding less than about 55 wt. % of the total solid additives added to form the second aqueous mixture. For example, adding a water soluble plasticizer to the second aqueous mixture may include adding less than about 50 wt. %, or less than about 40 wt. %, or less than about 30 wt. %, or less than about 25 wt. %, or between about 15 wt. % and about 55 wt. %, or between about 15 wt. % and about 50 wt. %, or between about 15 wt. % and about 40 wt. %, or between about 15 wt. % and about 30 wt. %, or between about 15 wt. % and about 25 wt. % of the total solid additives added to form the second aqueous mixture, and all values therebetween. As used herein, the term “total solid additives added to form the second aqueous mixture” refers to all of the components of the aqueous mixture except for water.

According to embodiments of the present disclosure, forming a second aqueous mixture may further include adding at least one emulsifying agent to the second aqueous mixture. Emulsifying agents as described herein may include, but are not limited to sodium dodecyl sulfate (SDS) and polysorbates, such as polyethylene glycol sorbitan monolaurate (Tween® 20) and polyoxyethylene sorbitan monooleate (Tween® 80) (each commercially available from Croda International, Snaith, United Kingdom).

According to embodiments of the present disclosure, forming a second aqueous mixture may further include adding at least one leachable solid to the second aqueous mixture. Leachable solids as described herein include materials that reinforce or create pores during the formation of the foam scaffold. Leachable solids may be, but are not limited to, nontoxic leachable materials such as salts, biocompatible mono and disaccharides and water-soluble proteins. Exemplary salts include, but are not limited to, sodium chloride, potassium chloride, calcium chloride, sodium tartrate, sodium citrate, and the like. Exemplary biocompatible mono and disaccharides include, but are not limited to, glucose, fructose, dextrose, maltose, lactose and sucrose. Exemplary water-soluble proteins include, but are not limited to, gelatin and agarose.

Each of the materials described above in relation to the second aqueous mixture may all be optionally added to the second aqueous mixture and may be added to the second aqueous mixture in any order with the possibility that two or more of the materials may be added to the second aqueous mixture simultaneously. In one exemplary method, forming a second aqueous mixture includes adding a leachable solid to an aqueous solution including a divalent metal salt and mixing the aqueous mixture to facilitate the dissolution of the leachable solid dissolves in the aqueous mixture. The at least one first polymer, the at least one second polymer and/or the water soluble plasticizer are subsequently added to the second aqueous mixture.

Methods for forming a dissolvable foam scaffold as described herein may further include, subsequent to forming the first and second aqueous mixtures, combining the second aqueous mixture with the first aqueous mixture to form a combined aqueous mixture. A foam may be formed from the combined aqueous mixture by introducing gas bubbles into the aqueous mixture through mixing, beating, agitating, aerating, whipping, injecting or other mechanical actions. The gas may be for example, but not limited to, air, nitrogen, helium, hydrogen, argon, carbon dioxide or other inert gas. Introducing gas bubbles into the combined aqueous mixture may then be done for a period of less than about 30 minutes, for example, between about 1 minute and about 30 minutes, or between about 3 minutes and about 25 minutes, or even between about 5 minutes and about 20 minutes. While introducing gas bubbles into the combined aqueous mixture, the method for forming a dissolvable foam scaffold may further include adding a gel inducing agent to the combined aqueous mixture. The gel inducing agent may be an acid that provides a buffering action and/or materials that slowly generate acid. Exemplary acids include, but are not limited to, lactic acid lactone, glycolic acid lactone, glucono delta lactone and acid anhydrides.

Methods for forming a dissolvable foam scaffold as described herein may further include, coating the dissolvable foam scaffold with an adhesion polymer coating. Coating the dissolvable foam scaffold may include exposing the scaffold to an aqueous solution having an adhesion polymer in the aqueous solution. As previously discussed, the adhesion polymer may include peptides. Exemplary peptides may include, but are not limited to BSP, vitronectin, fibronectin, laminin, Type I and IV collagen, denatured collagen (gelatin), and like peptides, and mixtures thereof. Additionally, the peptides may be those having an RGD sequence. The coating may be, for example, Synthemax® II-SC (commercially available from Corning, Incorporated, Corning, NY).

According to embodiments of the present disclosure, methods for culturing cells and spheroids in dissolvable and non-dissolvable foam scaffolds as described herein are also disclosed. Any type of cell or spheroid may be cultured in the foam scaffolds including, but not limited to, immortalized cells, primary culture cells, cancer cells, stem cells (e.g., embryonic or induced pluripotent), etc. The cells may be mammalian cells, avian cells, piscine cells, etc. The cells may be of any tissue type including, but not limited to, kidney, fibroblast, breast, skin, brain, ovary, lung, bone, nerve, muscle, cardiac, colorectal, pancreas, immune (e.g., B cell), blood, etc. The cells may be seeded into the foam scaffolds in any cultured form including disperse (e.g., freshly seeded), confluent, 2-dimensional, 3-dimensional, spheroid, etc. Culturing cells or spheroids in a foam scaffold may include seeding cells in the foam scaffold. Seeding cells in a foam scaffold may include contacting the scaffold with a solution containing the cells. During seeding cells in the foam scaffold, the cells enter the pores of the foam scaffold.

Culturing cells and spheroids in the foam scaffolds may further include contacting the scaffolds with cell culture medium. Generally, contacting the scaffolds with cell culture medium includes placing cells to be cultured in the scaffolds in an environment with medium in which the cells are to be cultured. Contacting the scaffolds with cell culture medium may include pipetting cell culture medium onto the scaffolds, or submerging the scaffolds in cell culture medium, or passing cell culture media over the scaffolds in a continuous manner. Generally, as used herein, the term “continuous” refers to culturing cells with a consistent flow of cell culture medium into and out of the cell culture environment. Such passing cell culture media over the scaffolds in a continuous manner may include submerging the scaffolds in cell culture medium for a predetermined period of time, then removing at least some of the cell culture medium after the predetermined period of time and adding fresh cell culture medium such that the volume of cell culture medium in contact with the dissolvable foam scaffold remains substantially constant. Cell culture medium may be removed and replaced according to any predetermined schedule. For example, at least some of the cell culture medium may be removed and replaced every hour, or every 12 hours, or every 24 hours, or every 2 days, or every 3 days, or every 4 days, or every 5 days. Cells may be cultured on the order of hours, days or weeks. For example, cells may be cultured for 12 hours, 18 hours, 24 hours, 2 days, 4 days, 6 days, 1 week, 2 weeks or even greater amounts of time. It should be understood that these are examples of culture times only and that any predetermined amount of time is acceptable.

Cell culture medium may be for example, but is not limited to, sugars, salts, amino acids, serum (e.g., fetal bovine serum), antibiotics, growth factors, differentiation factors, colorant, or other desired factors. Exemplary cell culture medium includes Dulbecco's Modified Eagle Medium (DMEM), Ham's F12 Nutrient Mixture, Minimum Essential Media (MEM), RPMI Medium, Iscove's Modified Dulbecco's Media (IMDM) Mesencult™-XF medium, RoosterNourish-MSC, RoosterNourish-MSC XF and the like. Other exemplary cell culture mediums include those tailored for collecting byproducts of cell cultures, such as defined, low particle medias including extracellular matrix collection medias such as RoosterCollect™-EV media, RoosterCollect™-EV-CC media, RoosterBio M2001 media, and the like.

According to embodiments of the present disclosure, methods for harvesting cells (including three-dimensional cell structures) from the foam scaffolds as described herein are also disclosed. In embodiments with a dissolvable foam scaffold, methods for harvesting cells as described herein may include digesting the dissolvable foam scaffold by exposing the dissolvable foam scaffold to an enzyme. As previously discussed, non-proteolytic enzymes suitable for digesting the foam scaffolds, harvesting cells, or both, include pectinolytic enzymes or pectinases, which are a heterogeneous group of related enzymes that hydrolyze the pectic substances, and alginate lyase. Commercially available sources of pectinases are generally multi-enzymatic, such as Pectinex™ ULTRA SP-L (commercially available from Novozyme North American, Inc., Franklinton, NC), a pectolytic enzyme preparation produced from a selected strain of Aspergillus aculeatus. Pectinex™ ULTRA SP-L contains mainly polygalacturonase, (EC 3.2.1.15) pectintranseliminase (EC 4.2.2.2) and pectinesterase (EC 3.1.1.11). Alginate lyase (EC 4.2.2.3) breaks down alginate and alginic acid and is commercially available from Sigma-Aldrich®, among others. The EC designation is the Enzyme Commission classification scheme for enzymes based on the chemical reactions the enzymes catalyze.

Exposing the dissolvable foam scaffold to an enzyme may include exposing the scaffold to enzyme concentrations of between about 1 and about 200 U. For example, the method may include exposing the scaffold to enzyme concentrations of between about 2 U and about 150 U, or between about 5 U and about 100 U, or even between about 10 U and about 75 U, and all values therebetween.

Methods for harvesting cells as described herein may further include exposing the material to a chelating agent. Exemplary chelating agents include, but are not limited to, ethylenediaminetetraacetic acid (EDTA), cyclohexanediaminetetraacetic (CDTA), ethylene glycol tetraacetic acid (EGTA), citric acid and tartaric acid. Exposing the dissolvable foam scaffold to a chelating agent may include exposing the scaffold to chelating agent concentrations of between about 1 mM and about 200 mM. For example, the method may include exposing the scaffold to chelating agent concentrations of between about 10 mM and about 150 mM, or between about 20 mM and about 100 mM, or even between about 25 mM and about 50 mM, and all values therebetween.

According to aspects of embodiments of this disclosure, the foam scaffolds of the present disclosure (dissolvable or non-dissolvable) can be used in a perfusion bioreactor for three-dimensional cell cultures (e.g., spheroids and organoids), and for production of cell culture by-products such as extracellular vesicles and extracellular matrix proteins. The perfusion bioreactors of this disclosure have an inlet that lets fluid into the bioreactor and an outlet that lets fluid out of the bioreactor. The perfusion bioreactors of the present disclosure also have a substrate (i.e. the porous scaffold) inside the bioreactor to assist in the culturing cells. Cells of a desired cell type may be added (known as “seeding”) directly in the foam scaffold before the foam scaffold is placed in the bioreactor to culture the cells. Alternatively, cells of a desired cell type may be added through the inlet (a location on the bioreactor where fluid may enter the bioreactor) to reach a foam scaffold inside of the bioreactor. With either option, the added cells will enter the pores and interconnected spaces between the pores (called “interconnects”) that are present in the foam scaffold. The pores and interconnects of the foam scaffold house the cells and allow the formation of spheroids, organoids, or other three-dimensional cell types. In some embodiments, at least 70%, 80%, 90%, or greater of the cells in the pores of the porous scaffold aggregate to form spheroids. In one specific embodiment, at least 80% of the cells in the pores of the porous scaffold aggregate to form spheroids. The formed spheroids, organoids, or other three-dimensional cell types remain contained by the pores and interconnects during cell culture in the bioreactor. For three-dimensional cell cultures, the foam scaffold is preferably non-adherent to the cells to provide optimal formation of the spheroids, organoids, or other three-dimensional cell culture types. In general, the less the adherence is to the foam scaffold, the more likely it is the cells will adhere to each other to form the three-dimensional culture. The culturing three-dimensional cells can produce a variety of byproducts, including extracellular vesicles, proteins, and other secreted materials. Reference to any particular byproduct (e.g., EVs) is not intended to limit the scope of embodiments with respect to that particular byproduct, which is used for illustration purposes only. It should be understood that exosomes and other cellular byproducts secreted by spheroids can be cultured with the scaffolds of the present disclosure instead of the EVs that have been described herein.

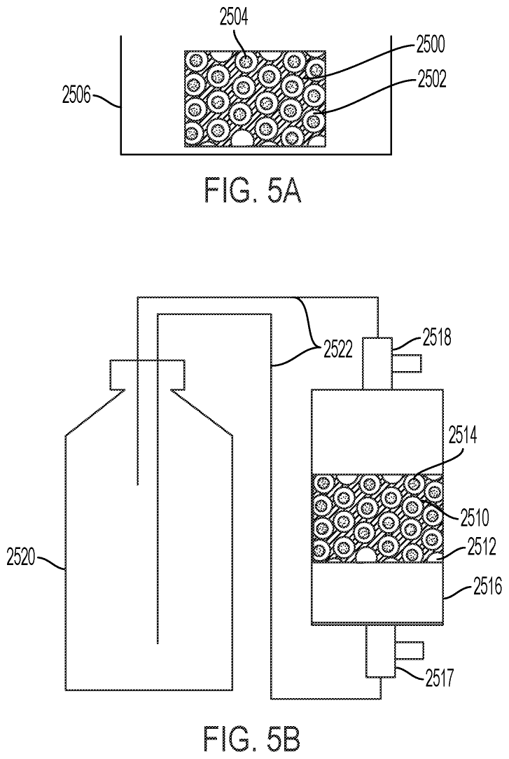

FIGS. 5A-B show illustrative examples of foam (porous) scaffolds used for three-dimensional cell cultures. FIG. 5A shows a porous scaffold made from a scaffold material 2500 with pores 2502 formed in the scaffold material 2500. The scaffold is placed in a tissue culture plate 2506 and cells are seeded onto it. Following a culture period, the cells form spheroids 2504 in the pore 2502. FIG. 5B shows a similar scaffold for growing spheroids, but this time the scaffold material 2510 having pores 2512 is placed in a bioreactor 2516 of a perfusion bioreactor system. The cells are seeded into the bioreactor 2516 through inlet 2517 to form spheroids 2514 in the pores 2512. The bioreactor 2516 has an outlet 2518 through which spent fluid may exit the bioreactor. The bioreactor system shown in FIG. 5B is a simplified depiction of such a system having a media conditioning vessel 2520 and a fluid flow path 2522 allowing perfusion and/or recirculation of media through the bioreactor 2516. Using the porous scaffold inside a bioreactor with media perfusion can increase in extracellular vesicle production. The system shown in FIG. 5B is an example only and is not intended to limit the scope of embodiments of this disclosure. For example, cells could instead be added to the foam scaffold 2500 through outlet 2518 of the bioreactor and fluid could exit through inlet 2517 of the bioreactor. As another example, a media feed vessel may be added to the perfusion bioreactor system that conditions the media conditioning vessel. As yet another example, pumps or controllers, or both, may exist on lines between the bioreactor and the media conditioning vessel.

According to aspects of embodiments of this disclosure, two-dimensional and three-dimensional cell structures (spheroids, organoids, and the like), and byproducts of these cells and structures, are cultured and harvested with the foam scaffolds of the present disclosure. The increase in cells from the time cells are seeded to the time they are harvested may be from about 0.2-fold to about 200-fold. In one embodiment, the increase in cells from a primary cell line (e.g., hMSC cells and the like) is between about 0.2-fold to about 100-fold, between about 0.2-fold and about 50-fold, between about 0.2-fold and about 30-fold, between about 0.2-fold and about 10-fold, or between about 0.5-fold and about 5-fold. In another embodiment, the increase in cells from a non-primary cell line (e.g., HEK293T cells and the like), is between about 5-fold and about 200-fold, between about 50-fold and about 150-fold, between about 50-fold and about 100-fold, between about 5-fold and about 50-fold, between about 7-fold and about 40-fold, or between about 8-fold and about 30-fold. In one specific embodiment, the increase in cells from a primary cell line is between about 0.2-fold and about 5-fold, five days after seeding cells in a foam scaffold and culturing the cells in a perfusion bioreactor. In another specific embodiment, the increase in cells from a non-primary cell line is between about 8-fold and about 30-fold, seven days after seeding cells in the foam scaffold and culturing the cells in a perfusion bioreactor. In another specific embodiment, the cell increase from a non-primary cell line is between about 50-fold and about 100-fold, one day after seeding cells in a foam scaffold and culturing the cells in a perfusion bioreactor. The times after cell seeding and the increases in fold after cell seeding with the foam scaffolds are exemplary in nature, other cell seeding times and increases in fold are contemplated.

When cells are introduced to foam (porous) scaffolds in bioreactors through perfusion, the capture of cells in the foam scaffold may be at least 1% of the cells added per pass through the bioreactor or more and may accumulate with additional passes through the bioreactor. For example, the capture of cells per pass in the foam scaffold may be at least 1%, 5%, 10%, 15%, 20%, 25% 30%, 35%, 40%, 45%, 50%, 55%, 60%, 65%, 70%, 75%, 80%, 85%, 90%, 95%, or greater of the cells added (or any value in between). In one embodiment, the capture of cells in the foam scaffold is at least 5% of cells being added after a single pass through the bioreactor. In another embodiment, the capture of cells in the foam scaffold is at least 10% of cells being added after a single pass through the bioreactor. Cells that exit the bioreactor in any one pass through the bioreactor may be recirculated back through the bioreactor for additional passes. The cells that have accumulated in the bioreactor at the end of all passes may be at least 5%, 10%, 15%, 20%, 25% 30%, 35%, 40%, 45%, 50%, 55%, 60%, 65%, 70%, 75%, 80%, 85%, 90%, 95%, or greater of the cells added (or any value in between). In one specific embodiment, the cells that have accumulated in the bioreactor at the end of all passes is at least 30%. In another specific embodiment, the cells that have accumulated in the bioreactor at the end of all passes is at least 40%. In yet another specific embodiment, the cells that have accumulated in the bioreactor at the end of all passes may be at least 50%.

In some embodiments, the addition of cells occurs at the inlet of the bioreactor. In some embodiments, the addition of cells occurs at a point before the inlet of the bioreactor. In one nonlimiting example, cells are added to a fluid line that feeds media to the bioreactor through a port on the fluid line. In another nonlimiting example, cells are added to a vessel containing media and then cells are perfused into the bioreactor at a set flow rate (for example, 500 mL/min, 200 mL/min, 50 mL/min, 20 mL/min, 10 mL/min, 5 mL/min, 1 mL/min, or 0.5 mL/min). In one embodiment, cells are perfused through the bioreactor at between about 0.1 mL/min and about 20 mL/min. In some embodiments, cells are perfused through the bioreactor at a linear velocity between about 2.3×10⁻⁶ m/s and about 1.2×10⁻³ m/s (Darcy velocity). As used herein, the term “linear velocity” refers to Darcy velocity. In some embodiments, cells are perfused through the bioreactor at a linear velocity (Darcy velocity) between about 1.0×10⁻⁵ m/s and about 5.0×10⁻⁴ m/s. In some embodiments, cells are perfused through the bioreactor at a linear velocity (Darcy velocity) between about 1.0×10⁻⁵ m/s and about 4.8×10⁻⁵ m/s, between about 1.0×10⁻⁵ m/s and about 2.4×10⁻⁴ m/s, or between about 1.0×10⁻⁵ m/s and about 3.6×10⁻⁴ m/s. In some embodiments, any cells that exit the bioreactor are recirculated back through the inlet of the bioreactor to attempt to capture the cells in another pass through the bioreactor.

It should be understood that the rate of perfusion does not need to be the same across the entire process. For example, while cells are in the process of seeding cells into a foam (porous) scaffold that exists in a bioreactor, a lower or higher flowrate may be used, such as 0.1 mL/min, 0.5 mL/min, 1 mL/min, 2 mL/min, 3 mL/min, 4 mL/min, or 5 ml/min, 10 mL/min, 15 mL/min, 20 mL/min, 50 mL/min, 100 mL/min, or some value between 0.1 mL/min to 100 mL/min. Likewise, a lower linear velocity (Darcy velocity) may be used during seeding of cells, such as (but not limited to) between about 1.0×10⁻⁵ m/s and about 4.8×10⁻⁵ m/s, between about 1.0×10⁻⁵ m/s and about 2.4×10⁻⁴ m/s, or greater than 0 m/s and less than 2.4×10⁻⁴ m/s After the foam scaffold has been seeded, a higher or lower flow rate may be used for culturing than was used for loading, such as 2 mL/min, 5 mL/min, 10 mL/min, 15 mL/min, 20 mL/min, 50 mL/min, 100 mL/min, 200 mL/min, 300 mL/min, 400 mL/min, 500 mL/min or some value between 2 mL/min and 500 mL/min. In a preferred embodiment, cell seeding occurs at a perfusion flow rate from about 0.1 mL/min to about 5 mL/min. In another preferred embodiment, cell culturing occurs at a perfusion flow rate from about 5 mL/min to about 15 mL/min. In some embodiments, media perfusion flow rate during cell seeding is at a linear velocity from about 2.3×10⁻⁶ m/s to about 1.2×10⁻³ m/s (Darcy velocity). In some embodiments, cells are perfused through the bioreactor at a linear velocity (Darcy velocity) between about 1.0×10⁻⁵ m/s and about 5.0×10⁻⁴ m/s. In some embodiments, cells are perfused through the bioreactor at a linear velocity (Darcy velocity) between about 1.0×10⁻⁵ m/s and about 4.8×10⁻⁵ m/s, between about 1.0×10⁻⁵ m/s and about 2.4×10⁻⁴ m/s, or between about 1.0×10⁻⁵ m/s and about 3.6×10⁴ m/s.

According to some embodiments, the flow of fluid through the scaffold during cell culturing is at a flow velocity in the range of about 2.3×10⁻⁶ m/s and about 1.2×10⁻³ m/s (Darcy velocity). In some embodiments, the fluid flows through the scaffold at a flow velocity in the range of 2.3×10⁶ m/s and about 1.2×10⁻⁵ m/s, about 1.2×10⁻⁵ m/s and about 5.0×10⁻⁴ m/s, or about 5.0×10⁻⁴ m/s and about 1.2×10⁻³ m/s (Darcy velocity). In some embodiments, the flow of fluid through the scaffold is at a linear velocity (Darcy velocity) between about 1.0×10⁻⁵ m/s and about 5.0×10⁻⁴ m/s. In some embodiments, cells are perfused through the bioreactor at a linear velocity (Darcy velocity) between about 1.0×10⁻⁵ m/s and about 4.8×10⁻⁵ m/s, between about 1.0×10⁻⁵ m/s and about 2.4×10⁴ m/s, or between about 1.0×10⁻⁵ m/s and about 3.6×10⁻⁴ m/s. The flow velocity of the medium through the scaffold is dependent on the dimension of the scaffold and the medium flow rate.

Fluid with a velocity can exert wall sheer stress on cells being cultured in porous scaffolds. For cell culture medium flowing in porous scaffolds with spheroids or other cells, the flow is generally laminar flow and the cell culture medium is a Newtonian fluid. According to some embodiments, shear stress stimulations on the spheroids and/or cells are generated by the movement of the media through at least one porous scaffold containing spheroids. In some embodiments, shear stress on the spheroids or cells is generated using a pump. In some embodiments, shear stress on the spheroids or cells is generated by any other method leading to movement of the media inside the scaffold including but not limited to agitation, vibration, rotating, waving, or tilting.

The values for shear stress can be determined by in silico modeling of the scaffold with computational fluid dynamics simulations. This was done for the scaffolds of this disclosure. A representative computer assisted drawing geometry of the scaffold was created through MATLAB coding and using the pore sizes, interconnect sizes and number of interconnects per pores measured on the scaffolds, creating a computational mesh. This computational mesh was then imported into ANSY Fluent for computational fluid dynamics simulations. The Reynolds numbers calculated were less than 1, which means the flow in the porous scaffolds is laminar. The shear stress provided to the cells and spheroids of the present disclosure were determined to be between 0 mPa and about 300 mPa. However, shear stress values greater than 300 mPa, such as 400 mPa, 500 mPa, 600 mPa, 700 mPa, 800 mPa, 900 mPa, or 1000 mPa, or greater, may be provided and the spheroids or cells may still retain their aggregation and/or still produce byproducts of interest.

According to some embodiments, the shear stress provided to the spheroids contained inside the scaffold's pores are between 0 mPa and about 300 mPa, or any value in between. In some embodiments, the shear stress provided to the spheroids contained inside the scaffold's pores are greater than 300 mPa. In some embodiments, the shear stress provided to the spheroids contained inside the scaffold's pores are greater than 0 mPa. In some embodiments, the shear stress provided to the spheroids contained inside the scaffold's pores is between 0.001 mPa and about 300 mPa, or any value in between. In yet other embodiments, the shear stress provided to the spheroids contained inside the scaffold's pores is between about 0.001 mPa and about 1 mPa, between about 1 mPa and about 50 mPa, between about 50 and about 150 mPa, or between about 150 mPa and 300 mPa, or any value in between these ranges.

According to some embodiments of this disclosure, byproducts are collected from culturing cells or three-dimensional cell structures with the foam (porous) scaffolds of the present disclosure, which may then be used for downstream applications. One such byproduct is extracellular vesicles (EVs). EVs are produced by cells that are cultured in the foam scaffolds of the present disclosure. Other byproducts that may be produced by perfusion with the foam scaffolds or methods described herein include but are not limited to, microvesicles, exosomes, other exosome-like particles, exomeres and other nano-particles, proteins, polypeptides, peptides, amino acids, lipids, polynucleotide sequences, and hormones. The byproducts may be genetically engineered or naturally occurring. The collection of the byproducts can be performed by any of the many methods known to those of ordinary skill the art.

Perfusion with the foam (porous) scaffolds of the present disclosure produce an increased number of byproducts compared to conventional 3D culturing methods and apparatuses. For vesicle particles, culturing spheroids by perfusion in the foam scaffolds of the present disclosure produces between about 1×10⁹ and about 1×10¹³ EVs per million cells (i.e., between about 1×10³ and about 1×10⁷ EVs per cell) after culturing cells for about 48 hours. In one embodiment, at least 1×10¹¹ EVs are produced for every 1×10⁶ cells of a cell type (i.e., at least 1×10⁵ EVs per cell) after about 48 hours of culturing time with a perfusion rate having linear velocity of about 2.4×10⁻⁴ m/s (Darcy velocity). In another embodiment, at least 1×10¹² EVs are produced for every 1×10⁶ cells of a cell type (i.e., at least 1×10⁶ EVs per cell) after about 48 hours of culturing time with a perfusion rate having linear velocity of about 2.4×10⁻⁴ m/s (Darcy velocity). In another embodiment, at least 1×10¹³ EVs are produced for every 1×10⁶ cells of a cell type (i.e., at least 1×10⁷ EVs per cell) after about 48 hours of culturing time with a perfusion rate having linear velocity of about 2.4×10⁻⁴ m/s (Darcy velocity). In yet another embodiment, between about 1×10¹¹ EVs and about 1×10¹³ EVs are produced for every 1×10⁶ cells of a cell type (i.e., between about 1×10⁵ and about 1×10⁷ EVs per cell of a cell type) after about 48 hours of culturing time with a perfusion rate having linear velocity of about 2.4×10⁻⁴ m/s (Darcy velocity). As used herein the term “extracellular vesicles” or “EVs” refers to membrane bound vesicles that are that are secreted from cells and can have been produced in the endosomal compartment. EVs, contain various molecular components from the cells. These components are named “cargo” and may have biological function. These cargos may be containing some or all of: proteins, lipids, mitochondrial components and genetic materials (RNA and/or DNA).

The EVs produced by cell cultures with the foam (porous) scaffolds of the present disclosure each have a size, called a vesicle diameter, and collectively the EVs have an average vesicle diameter. The size of the EVs can be determined by multi-angle dynamic light scattering (MADLS). In one embodiment, the EVs collected from cells cultured in foam scaffolds of the present disclosure have a diameter of between about 40 nm and about 200 nm, between about 50 nm and about 150 nm, or between about 60 nm and about 140 nm, between about 70 nm and about 130 nm, between about 80 nm and about 120 nm, or between about 90 nm and about 110 nm, when measured by MADLS. In another embodiment, the EVs collected from cells cultured in foam scaffolds of the present disclosure have an average diameter of between about 80 nm and about 120 nm, or between about 90 and about 110 nm, or between about 95 nm and about 105 nm, when measured by MADLS. In one specific embodiment, the EVs have an average diameter from about 100 nm to about 110 nm.

The EVs produced from cells cultured in foam scaffolds of the present disclosure also have a functionality level associated with them. Such functionality of EVs can be measured by a wound healing assay using HT-1080 cells (epithelial cells derived from connective tissue, ATCC, CCL-121) to determine directional cell migration in vitro. Wound healing assays of the present disclosure comprise creating a cell monolayer from HT-1080 cells, creating a wound in the cell monolayer, imaging the wound after it is formed, treating the wounds with 2×10⁹ EVs collected from a cell culture, and then imaging the treated wounds at regular intervals during cell migration to close the wound. The same process can be done for a control (no EVs added). A plot of the percent wound healing over time for each wound assay can then be created to compare the functionality of different EV batches or processes.

According to some embodiments, EVs collected from cell cultures in the foam scaffolds of the present disclosure have a similar or an improved functionality for wound healing than EVs collected from non-perfusion-based cell culture methods. In some embodiments the improved EV functionality is between about 2%-50% faster at wound healing from perfusion-based culturing than static culturing (i.e., in microwells of culture plates or culture flasks) after 6 hours post-wound formation in a monolayer of HT-1080 cells, when measured by a wound healing assay. In one specific embodiment, the improved EV functionality is between about 2%-10%, about 10%-20%, about 20%-30%, about 30%-40%, or about 40%-50% faster with EVs produced by perfusion-based culturing than EVs produced by static culturing as measured after 6 hours post-wound formation in a monolayer of HT-1080 cells, when measured by a wound healing assay. In some embodiments, the improved EV functionality is between about 2%-30% faster at wound healing than EVs produced by a non-perfusion-based cell culture method after 6 hours post-wound formation in a monolayer of HT-1080 cells, when measured by a wound healing assay. In one specific embodiment, the improved EV functionality is between about 2%-5%, about 5%-10%, about 10%-15%, about 15%-20%, about 20%-25%, or about 25%-30% faster than EVs produced by a non-perfusion-based cell culture method after 6 hours post-wound formation in a monolayer of HT-1080 cells, when measured by a wound healing assay.

### EXAMPLES

Embodiments of the present disclosure are further described below with respect to certain exemplary and specific embodiments thereof, which are illustrative only and not intended to be limiting.

Example 1

| Additive Component | Type of Additive | Amount of Additive | |
|---|---|---|---|
| Polysaccharide | 2.0 wt. % PGA | 162 | grams |
| Divalent Metal Salt | CaCO₃ | 1.06 | grams |
| Emulsifying Agent | TWEEN ® 20 | 0.125 | grams |
| Plasticizer | Glycerol | 7.5 | grams |
| Sugar | Sucrose | 17.5 | grams |
| Surface Active Polymer | Methocel HPMC | 1.94 | grams |
| | Culminal 724 | | |
| Surface Active Polymer | Porcine Gelatin | 0.97 | grams |
| Non-Surface Active Polymer | None | | |
| Gel Inducing Agent | GDL | 3.77 | grams |

A first aqueous mixture containing 2.0 wt. % polygalacturonic acid (PGA) was prepared by dissolving about 162 grams of polygalacturonic acid sodium salt in demineralized water in an oil bath set at a temperature of 104 °C. The aqueous mixture was cooled to room temperature. A second aqueous mixture was prepared by adding about 7.5 grams of glycerol to ultrapure water and heating under a microwave at 800W for about 30 seconds. About 1.06 grams of CaCO₃ and about 0.125 grams of TWEEN® 20 were added to the second aqueous mixture. The second aqueous mixture was then sonicated for about 1 minute and then transferred to the bowl of a KitchenAid mixer equipped with a wire loop whip. About 17.5 grams sucrose and about 1.94 grams Methocel HPMC Culminal 724 were then added to the bowl of the KitchenAid mixer and the aqueous mixture was stirred for about 5 minutes. The first aqueous mixture containing 2.0 wt. % PGA were added to form a combined aqueous mixture in the mixing bowl and mixed at a stir speed (speed 1 of the KitchenAid mixer) for about 3 minutes. The combined aqueous mixture was then whipped at a fast whipping speed (speed 10 of the KitchenAid mixer) for about 20 minutes to introduce air into the combined aqueous mixture. While continuing to whip the combined aqueous mixture, a solution of about 3.77 grams of gluconolactone (GDL) in about 30 mL of water was added to the mixing bowl and whipping was continued for about 1 minute.

An opaque white foam was obtained following the process discussed above. The foam was left uncovered in the mixing bowl at room temperature for about 1 hour to allow time for crosslinking to take place within the foam. The foam was then exposed to temperatures of about −80 °C for about 16 hours to freeze the foam and then exposed to a temperature of −86 °C and a pressure of 0.11 mbar for about 72 hours. The resulting foam was observed to have a wet foam density of about 0.21 g/cc and a dry foam density of about 0.06 g/cc and was observed to be porous with highly interconnected pores. FIG. 2 shows an SEM picture of the foam prepared in this Example 1.

### Example 2

Culturing Vero cells in a foam formed in accordance with the process of Example 4 was investigated. Vero cells (ATCCR CCL-81, commercially available from ATCC, Manassas, VA) were cultured on cell culture plates in IMDM medium supplemented with 10% fetal bovine serum (FBS). The foam was cut into portions that were about 2-3 mm thick and had diameters of about 22 mm. The foam portions were sanitized in 70% aqueous ethanol for about 5.0 minutes, then placed in separate wells of a 6-Well Ultra-Low Attachment Cell Culture Plate. The foam portions were washed twice in ultrapure water and once in IMDM medium. Excess medium was removed from the wells before seeding.

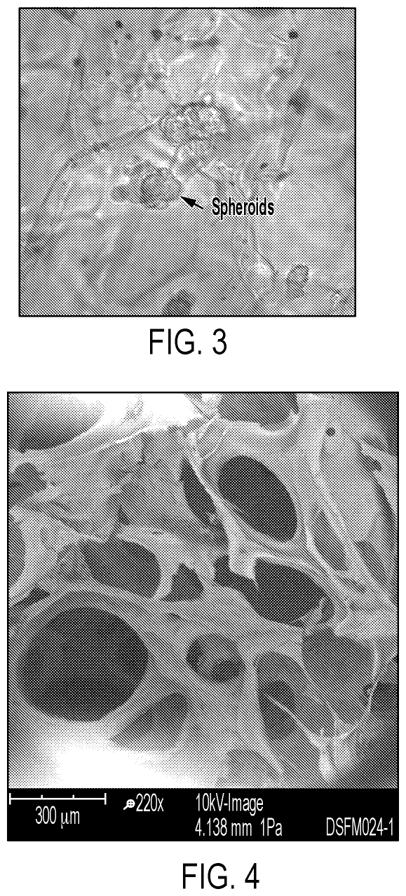

Vero cells were harvested from the cell culture plates using trypsin, re-suspended in IMDM medium and 150 μL containing about 100,000 cells were seeded in each of the foam portions which were positioned in the wells of the 6-Well Cell Culture Plate. The 6-Cell Culture Plate was placed in a cell culture incubator and, after about 2.0 hours, about 3.0 mL of IMDM medium was added to each well. After about 18 hours in the cell culture incubator, the foam portions were visualized using phase contrast microscopy. An image obtained from the phase contrast microscopy is depicted in FIG. 3 which shows that the cells did not adhere to the uncoated foam portions, but instead formed spheroids in the pores of the foam portions. As such, it was determined that the dissolvable foam scaffolds of the present disclosure may be utilized to culture spheroids or non-adherent cells.

### Example 3

Some of the dissolvable foams disclosed herein use various components (e.g., sucrose, glycerol, dextran, and others) that are added to facilitate the foaming process and account for a significant amount (e.g., by weight) of the solution used in the foaming process. For example, those components can take up to 90% of the final weight of the foam. Most of those components are not covalently associated with the foam structure and will dissociate from the foam during coating or may need to be removed before cell culture. In cell culture, if not removed, those materials can potentially change the cell media composition, block cell binding epitopes, and reduce cell attachment. After released into cell culture media, those materials can significantly change the media osmotic pressure. In the foaming process, surface active molecules or foaming agents, such as surfactants, are required. For example, Pluronic® P123 may be used in the foaming process, as disclosed in some of the below examples. However, the above examples also still use other components such as sucrose, dextran, glycerol, and Tween® 20 for foaming. To simplify the process, cut the cost production, and enable the direct use of the dissolvable foam in cell culture without removing extra components, it would be beneficial to eliminate those extra materials in the foam, as discussed in the following paragraphs.

In a foam, as in any porous material, pore size is important for the flow dynamics and total surface area. It has been observed that by using smaller size calcium carbonate particles as the source for gelation, much smaller size of pores was observed. It is believed that the smaller size calcium carbonate particles can lead to better distribution and easier release of calcium during mixing before acid being added, which increases the viscosity of the PGA solution. This leads to the foam cells being difficult to merge together to form large pores. However, faster release of calcium during foaming can also cause the PGA to become over-crosslinked before mixing completely and can damage the foam structure. It is desirable to be able to control pore size by control the viscosity of PGA solution during foaming but to also prevent uncontrolled over-crosslinking.

In this Example 3, the use of a plasticizer (e.g., glycerol), a foaming enhancement agent or sugar (e.g., sucrose), and a foaming enhancement agent in the form of a non-surface active polymer (e.g., dextran) are eliminated by using a high-HLB surface active polymer (such as, e.g., Poloxamer 407 or Pluronic® F127) in place of two surfactants used in previous examples (e.g., a surface active polymer such as P123 and an emulsifying agent such as Tween® 20). This provides a much simpler formulation for the dissolvable foam and eliminates ingredients that require further downstream processing or may potentially negatively impact cell culture. In addition, it is possible to control viscosity of the simplified formulation without adding any new components. This simplified formulation has the several advantages. For instance, the new formulation removes non-critical materials, which may in some cases be undesirable in the finished dissolvable foam product. This elimination of materials also greatly simplifies the process and reduces the cost. In addition, a large amount of leachable materials are eliminated from foam structure-materials which could otherwise potentially change cell media osmotic pressure and block cell binding epitopes during application. This will enable the elimination of a separate coating step in the future and enable direct usage of the foams in cell culture. Further, the simplified formulation provides tunable viscosity without adding additional components. This makes future optimization of the foaming process and control of foam structure easier and cheaper. Finally, the simplified formulation can benefit a continuous foaming process by using a shear thinning property of the material.

The foaming formulation according to an example embodiment from some the above examples (which for the remainder of this Example 3 will be referred to as Comparative Example) included a relatively long list of materials, as shown below in Table 1. In contrast, the simplified formulation of this Example 3 eliminates the plasticizer (e.g., glycerol), the emulsifying agent or second polymer having surface activity (e.g., Tween® 20), and other foaming enhancers or polymers having no surface activity (e.g., sucrose and dextran). The simplified formulation in Table 1 also uses a poloxamer (e.g., F127) of higher HLB than that of the P123 used in the comparative example (Pluronic® P123 has a HLB of 8 compared to the HLB of 22 for Pluronic® F127). Most of the additional ingredients in the Comparative Example were used to provide physical properties to enable the foaming process, as highlighted in the Table 1. These additional ingredients (Tween® 20, glycerol, sucrose, and dextran) account for about 80% of the total weight (excluding water) of the formulation in the Comparative Example. Table 2 below compares the weight percentages of the components in the Comparative Example and Simplified Formulation of Table 1. Those materials may have no value for final product and may need to be removed during a subsequent coating process. If the foam article is used in cell culture, those materials need be removed to prevent significant change of osmotic pressure or obstruction of cell binding epitopes.

TABLE 1

| | | | Simplified | | |
|---|---|---|---|---|---|
| | Comparative | | Formulation | | |
| | Example | | (Example 3) | | |
| Component | Amounts | | Amounts | | Comment |
| PGA | 3.24 | g | 3.24 | g | Base material of foam (polysaccharide) |
| F127/P123 | 0.97 g | | 0.97 g | | Polymer surfactant (surface active polymer) |
| | (P123) | | (F127) | | |
| Tween ® 20 | 115 μL | | 0 | mg | High HLB surfactant (emulsifying agent) |
| | (126.5 mg) | | | | |
| Glycerol | 6 ml | | 0 | g | Viscosity adjustment in foaming and plasticizer |
| | (7.56 g) | | | | (plasticizer) |
| Sucrose | 17.5 | g | 0 | g | Foaming enhancement (sugar) |
| Dextran | 9.7 | g | 0 | g | Foam enhancement (non-surface active polymer) |
| CaCO3 | 0.53 | g | 0.53 | g | Calcium source for crosslinking (divalent metal |
| | | | | | salt) |
| GDL | 3.77 | g | 0.5 g + 3.5 g | | Acid for calcium release (gel inducing agent) |
| Total weight | 43.4 | g | 8.7 | g | |

TABLE 2

| | Comparative | Simplified Formulation |
|---|---|---|
| Component | Example (wt. %) | (Example 3) (wt. %) |
| PGA | 7.47 | 37.07 |
| F127/P123 | 2.24 | 11.10 |
| Tween ® 20 | 0.29 | 0.00 |
| Glycerol | 17.42 | 0.00 |
| Sucrose | 40.33 | 0.00 |
| Dextran | 22.35 | 0.00 |
| CaCO3 | 1.22 | 6.06 |
| GDL | 8.69 | 45.77 |
| Total | 100 | 100 |

In this Example 3, Pluronic® F127 can replace a mixed surfactant of Pluronic® P123 and Tween® 20. It also enables good foaming without adding any sucrose, glycerol, or dextran. Measurements showed that the desired amount of porosity in the foam can be comparable to that of the earlier examples disclosed herein (including the Comparative Example). FIG. 4 shows an SEM image of a foam formed from the simplified formulation of Example 3, which has comparable pore structure as the foam made from the other Examples herein with more complex formulations.

Replacing P123 with F127 also benefits the process. P123 is a paste material which is difficult to aliquot. It is also difficult to be dissolved in water and the dissolving process can take overnight. F127 is powder which can be easily weighed out during formulation and the dissolution can be completed in one hour. Those properties can benefit manufacturing process.

By adding a small amount of calcium ion in PGA solution, the PGA molecules can be partially crosslinked and the viscosity of the solution increased. This enables creation of a shear-thinning and dissolvable material for 3D bioprinting. When amount of calcium ion was added to bind to about 10% of carboxyl groups in the PGA molecules, the solution increased viscosity by 1000× with shear rate of l/s, which was about 10 times of the glycerol (1.4×10³ mPa*s). When exposed to 1000 l/s shear rate, the viscosity drops to 1/100 and became 1/10 of the glycerol. The reduction of viscosity can be recovered quickly after the shear force removed. By adjusting the amount of calcium, it is possible to further tune the range of viscosity and shear-thinning response.

Thus, according to embodiments of the simplified formulation, a small amount of calcium can be added to increase the viscosity of the PGA solution before the start foaming. This can help increase the viscosity and provide shear thinning attributes. Under shear of mixing blades, the solution reduces viscosity which helps incorporate air bubble into the form. When mixing is stopped, the solution viscosity quickly increases which helps stabilize the foam and prevent liquid drainage. This can further eliminate the need of foaming stabilizer materials such as glycerol, sucrose, and Dextran. By controlling the solution viscosity, the speed of bubble merging can be increased or decreased, and it is possible to change the pore size in the foam. The shear thinning property can also benefit a continuous foaming process. After the foam is extruded from a continuous foaming mixer, it can quickly stabilize the pore structure. To do partial crosslinking, instead of adding all of the GDL at the end of the foaming step, part of the GDL can be introduced before foaming (e.g., the 0.5 g in Table 1), which leads to partial release of calcium from calcium carbonate and partially crosslink of PGA. At end of foaming step, the rest of the GDL can be added (e.g., the 3.5 g in Table 1) or an acid molecule such as acetic acid can be introduced in the form of a vapor to complete the gelation process.

According to aspects of embodiments of this disclosure, spheroids can be formed by cells entering the scaffold either by seeding the cells directly on top of the scaffold in a tissue culture plate (FIG. 5A) or through perfusion in a bioreactor (FIG. 5B). Cells will enter the porous material and be “trapped” or confined within the pores. As the cells will not be able to adhere to the material, they will adhere to each other to form spheroids. The cells or spheroid can produce a variety of byproducts, including EVs, proteins, and other secreted materials. Reference to any particular byproduct (e.g., EVs) is not intended to limit the scope of embodiments with respect to that particular byproduct, which is used for illustration purposes only. FIG. 5A shows a porous scaffold made from a scaffold material 2500 with pores 2502 formed in the scaffold material 2500. The scaffold is placed in a tissue culture plate 2506 and cells are seeded onto it. Following a culture period, the cells form spheroids 2504 in the pore 2502. FIG. 5B shows a similar scaffold for growing spheroids, but this time the scaffold material 2510 having pores 2512 is placed in a bioreactor 2516 of a perfusion bioreactor system. The cells are seeded into the bioreactor 2516 to form spheroids 2514 in the pores 2512. The bioreactor system shown in FIG. 5B is a simplified depiction of such a system having a media conditioning vessel 2520 and a fluid flow path 2522 allowing perfusion and/or recirculation of media through the bioreactor 2516. Using the porous scaffold inside a bioreactor with media perfusion can increase extracellular vesicle production. The rate of media perfusion through the bioreactor will vary based on the application, cell type, and other factors. Some examples of embodiments may use a media perfusion having a flow rate of from about 0.5 mL/min to about 50 mL/min. The system shown in FIG. 5B is an example only and is not intended to limit the scope of embodiments of this disclosure. Cells will enter the porous material and be “trapped” or confined within the pores. As the cells will not be able to adhere to the material, they will adhere to each other to form spheroids.

According to embodiments, the scaffold is dissolvable and therefore the spheroids can be readily recovered from the scaffold and used for other applications, such as toxicity assays or other applications that would be appreciated by a person of ordinary skill in the art. To allow spheroid formation and EV production, any type of scaffold material that is made from a biocompatible material with controlled porosity and that does not allow cell adhesion can be used. To recover spheroids, the scaffold material is made from a biocompatible, digestible material with controlled porosity and that does not allow cell adhesion. The porosity and structure of the porous scaffold can be controlled. In some preferred embodiments, the porous scaffold comprises of scaffold with high porosity and large pores but with smaller interconnects of scaffold material.

### Example 4

This example compares different scaffold characteristics based on their composition. These data looks at the impact certain reagents have on pore or interconnect size or number and how then these specific characteristics impact cell proliferation, spheroid maintenance in the foam or cell production, among other parameters.

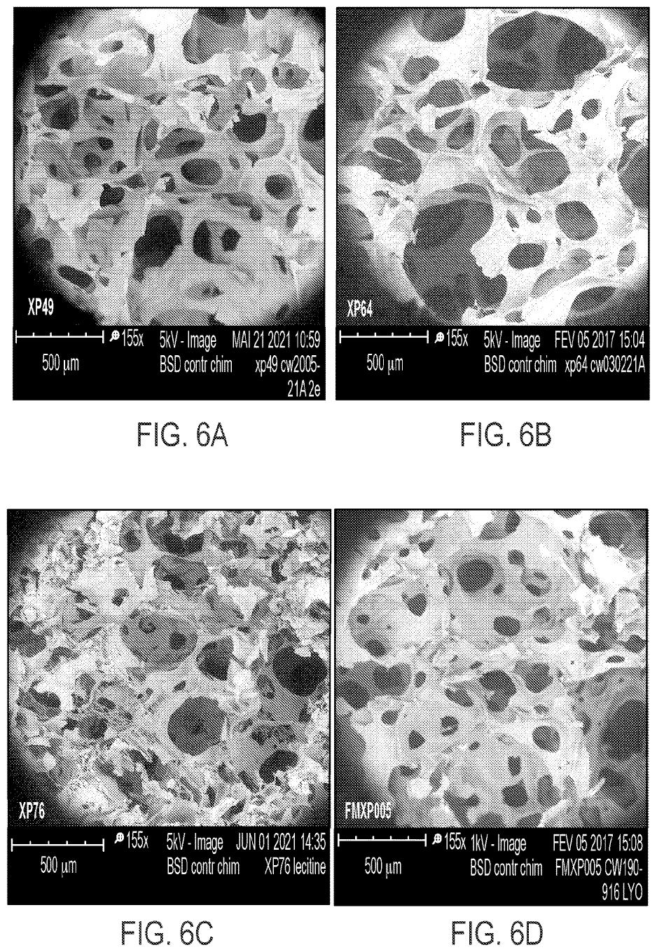

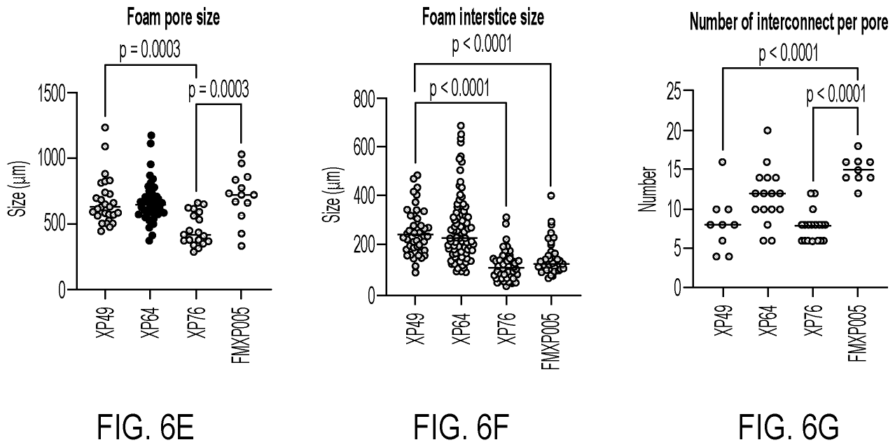

Four different compositions based on 2.0 wt. % PGA were assessed: XP49, XP64, XP76 and FMXP005. XP49 (FIG. 6A) uses Tween® 20 as the only emulsifying agent, Pluronic® P123 as a surface-active polymer, and dextran as a non-surface-active polymer (composition is in Table 2).

TABLE 2
XP49 Composition

| Additive Component | Type of Additive | Amount of Additive | |
|---|---|---|---|
| Polysaccharide | 2.0 wt. % PGA | 162 | grams |
| Divalent Metal Salt | CaCO3 | 0.53 | grams |
| Emulsifying Agent | TWEEN ® 20 | 0.125 | grams |
| Plasticizer | Glycerol | 7.5 | grams |
| Sugar | Sucrose | 17.5 | grams |
| Surface Active Polymer | Pluronic ® P123 | 0.194 | grams |
| Non-Surface Active Polymer | Dextran | 1.75 | grams |
| Gel Inducing Agent | GDL | 7.54 | grams |

XP64 (FIG. 6B) uses both Tween® 20 and SDS as emulsifying agents, Pluronic® P123 as a surface-active polymer, and dextran as a non-surface-active polymer (composition is in Table 3).

TABLE 3
XP64 Composition

| Additive Component | Type of Additive | Amount of Additive | |
|---|---|---|---|
| Polysaccharide | 2.0 wt. % PGA | 162 | grams |
| Divalent Metal Salt | CaCO3 | 0.53 | grams |
| Emulsifying Agent | TWEEN ® 20 | 0.125 | grams |
| Emulsifying Agent | SDS | 0.248 | grams |
| Plasticizer | Glycerol | 7.5 | grams |
| Sugar | Sucrose | 17.5 | grams |
| Surface Active Polymer | Pluronic ® P123 | 0.97 | grams |
| Non-Surface Active Polymer | Dextran | 0.97 | grams |
| Gel Inducing Agent | GDL | 3.77 | grams |

XP76 (FIG. 6C) uses soy lecithin as the only emulsifying agent and Methocel HPMC Culminal 724 as a surface-active polymer. It does not contain any non-surface-active polymer (composition is in Table 4).

TABLE 4
XP76 Composition

| Additive Component | Type of Additive | Amount of Additive | |
|---|---|---|---|
| Polysaccharide | 2.0 wt. % PGA | 162 | grams |
| Divalent Metal Salt | CaCO3 | 0.53 | grams |
| Emulsifying Agent | soy lecithin | 0.248 | grams |
| Plasticizer | Glycerol | 7.5 | grams |
| Sugar | Sucrose | 17.5 | grams |
| Surface Active Polymer | Methocel HPMC | 1.94 | grams |
| | Culminal 724 | | |
| Gel Inducing Agent | GDL | 3.77 | grams |

FMXP005 (FIG. 6D) uses SDS as the only emulsifying agent and dextran as a non-surface-active polymer. It does not contain any surface-active polymer (composition is in Table 5).

TABLE 5
FMXP005 Composition

| Additive Component | Type of Additive | Amount of Additive | |
|---|---|---|---|
| Polysaccharide | 2.0 wt. % PGA | 162 | grams |
| Divalent Metal Salt | CaCO3 | 0.53 | grams |
| Emulsifying Agent | SDS | 0.025 | grams |
| Plasticizer | Glycerol | 7.5 | grams |
| Sugar | Sucrose | 17.5 | grams |
| Surface Active Polymer | N/A | N/A | |
| Non-Surface Active Polymer | Dextran | 0.97 | grams |
| Gel Inducing Agent | GDL | 3.77 | grams |

Scaffolds for each of the XP49, XP64, XP76, and FMXP005 compositions were produced, dried and sliced for analysis by microscopy. Three parameters were quantified by scanning electron microscopy: pore size, interconnect (passage) size and number of interconnects (passages) per pore.

For all compositions, as shown in FIG. 6E the distribution of pore diameters (i.e., the maximum distance across a cross sectional area of a pore) for each composition was relatively wide. However, pore diameters from XP49 and XP64 were largely between 450 μm and 850 μm in diameter in size. The XP76 scaffold resulted in the smallest pores diameters, with pore diameters largely between 300 μm and 650 μm in diameter. The FMXP005 scaffold resulted in the largest pore diameter distribution, with pore diameters largely between 350 μm and 1000 μm. The distribution of pore diameter across all four foam compositions was largely between 300 μm and 1000 μm.

The distribution of interconnect diameters (i.e., the maximum distance across a cross-sectional area of a passage space that links pores together) had similarities to the distribution of pore diameters across the four compositions (FIG. 6F). Both XP49 and XP64 interconnect sizes were largely between 150 μm to 500 μm in diameter. XP76 had the smallest interconnect diameters of the compositions, with diameters largely between 30 μm and 300 μm. Even though the FMXP005 composition had the largest pore diameters, it did not have the largest interconnect diameters, with diameters largely between 70 μm and 400 μm.

The number of interconnects (passages) per pore was also investigated (FIG. 6G). The addition of SDS as an emulsifying agent in the XP64 scaffold, seems to affect the number of interconnects per pores compared to the XP49 scaffold. The number of interconnects per pore for XP64 was largely between 6 and 20 (median at 12) interconnect per pores, whereas XP49 was largely between 4 and 16 (median at 8) interconnect per pore. For XP76, the number of interconnects per pore were largely between 6 and 12 (median at 8) interconnect per pore. For FMXP005, the number of interconnects per pore was larger, resulting in between 12 and 18 (median at 15) interconnects per pore.

In addition, the density of the dry scaffolds were also measured. XP49 scaffold presented a density of about 0.038 to 0.046 g/cm³, XP64 presented a density of about 0.029 to 0.030 g/cm³, XP76 presented a density of about 0.071 to 0.09 g/cm³ and FMXP005 presented a density of about 0.048 to 0.057 g/cm³.

Taken together these data indicate that the scaffold composition can impact the pore diameter, interconnect diameter and the number of interconnects per pore as shown above.

### Example 5

In this Example, spheroid formation was demonstrated in the scaffold in a tissue culture plate using either an immortalized cell line (HEK293T cells) or a primary cell line (bone marrow-derived human mesenchymal stem cells, hMSC). The foam scaffolds had a cylinder shape with a diameter of 1 cm and a thickness of 0.4 cm. Two different compositions (XP64 and XP76)—leading to different pore and interconnect sizes as described in Example 4—were tested.

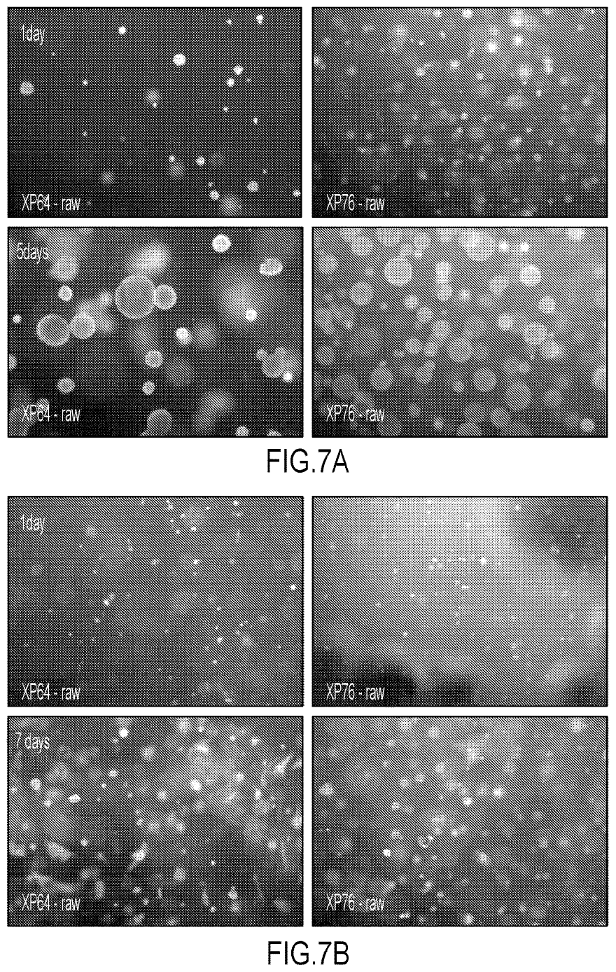

FIGS. 6B and 6C show example photographs of the uncoated foam scaffolds. After foam preparation, the scaffolds were washed with sterile filtered demineralized water and sterilized by 70% ethanol. Scaffolds were added into the wells of an Ultra-Low Attachment 6-well plate and the appropriate cell culture media was then added to the well. The plate was transferred into a cell culture incubator set at 37 °C, 5% CO₂ for equilibration. Cells were dissociated from their culture flask by either Tryspin (HEK293T) or TrypLE (hMSC) and the concentration was adjusted to 1×10⁶ cells/mL. To seed the foam scaffolds, the media was first removed from the well and 150 μL of the cell suspension added on the top of the foam for a total of 1.5×10⁵ cells/scaffold. The plate was transferred back into the cell culture incubator for 2 hours before adding 3 mL of the appropriate media into each well without perturbing the scaffolds. At one day after seeding, the initial spheroid formation was assessed by staining the live cells with Calcein AM. Spheroids were observed in all conditions and for both cell types (FIGS. 7A (HEK293T cells) and 7B (hMSC), first rows). Interestingly, there were more spheroids in the XP76 scaffold compared to the XP64 scaffold indicating a better cell maintenance in the XP76 scaffold. This could be due to the smaller interconnect and/or the smaller number of interconnects per pore in the XP76 compared to the XP64 scaffold as described in Example 4. At 7 days after seeding, the spheroid growth was assessed, as shown in the photographs in the bottom rows of FIGS. 7A (HEK293T cells) and 7B (hMSC). In all conditions, spheroids were larger at day 7 than at day 1, especially with the HEK293T cells. Cell counts after scaffold digestion and spheroid dissociation indicated similar results.

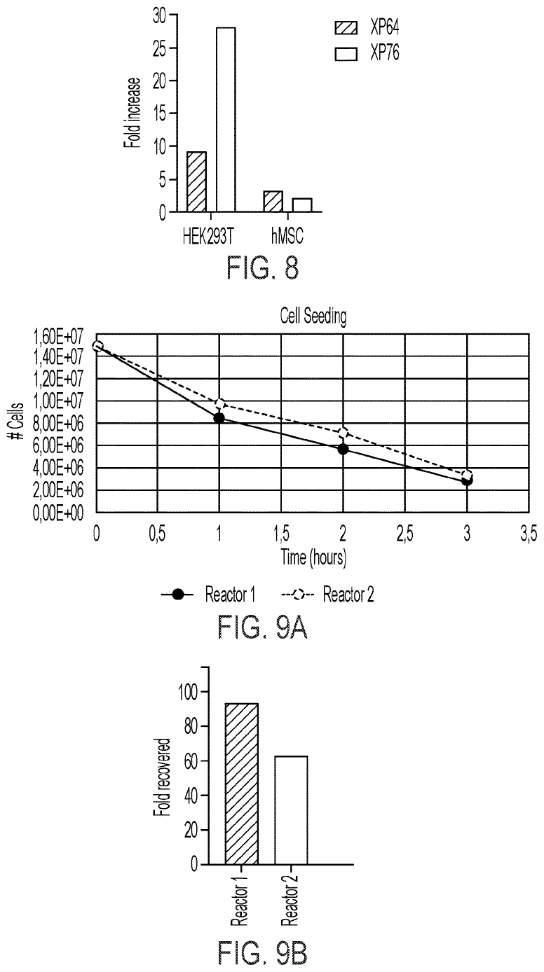

FIG. 8 is a bar graph showing the cell counts after scaffold digestion and spheroid dissociation in terms of the fold increase in cells after seeding for both experiments involving the HEK293T cells and the hMSC cells. The data shows cell growth at either five days after seeding for HEK293T cells or seven days after seeding for hMSC cells. The data clearly indicates spheroid and/or cell growth in the foam scaffold. The increase in the amount of cells for the HEK29T cells was about 9-fold to about 27-fold, while the increase for the hMSC cells was about 2— to 3-fold. Regarding HEK293T cells, the low cell growth numbers in XP64 scaffold appears to mainly be due to the loss of cells during seeding because of the larger interconnect and/or interconnects per pores compared to the XP76 scaffold. Growth of hMSC cells therefore remained limited in both scaffolds as expected for these cells.

### Example 6

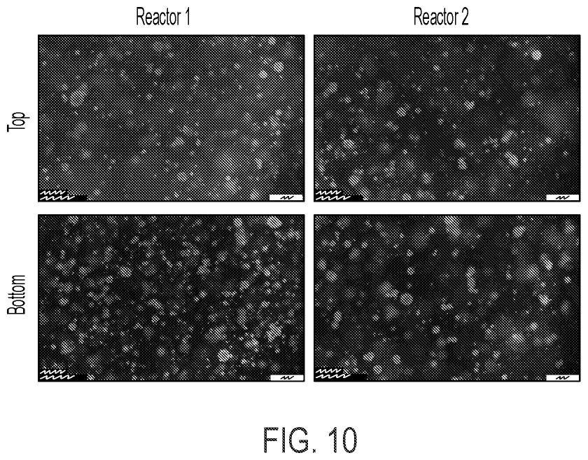

As discussed above, cells can also be seeded into a porous (foam) scaffold within a bioreactor under perfusion conditions, as shown in FIG. 5B. In this example, an immortalized cell line (HEK293T cells) was seeded into two of such perfusion bioreactors containing uncoated dissolvable foam scaffolds. The foam scaffolds had a cylinder shape with a diameter of 3 cm and a thickness of 1 cm. After preparation, the dry scaffolds were placed into the bioreactor cartridge. The scaffolds were then washed with sterile filtered demineralized water and sterilized inside the bioreactor through perfusion. The appropriate cell culture media was then added to a media stock bottle and perfused through the scaffold. The bioreactor was transferred into a cell culture incubator set at 37 °C, 5% CO₂ for equilibration for 1 hour. Cells were dissociated from their culture flask by Tryspin 0.25% and their concentration was adjusted to 1×10⁶ cells/mL. To seed the scaffolds, 10 mL of the cell suspension was added into the media stock bottle for a total of 10×10⁶ cells/scaffold. The perfusion flow rate was adjusted to 10 mL/min for cell seeding and cell seeding was monitored for 3 hours, as shown in FIG. 9A, before decreasing the media flow rate to 1 mL/min. FIG. 9A shows a plot of the number of cells counted in the recirculating media over time—the downward sloping line representing a decrease in HEK293T cells in the media over time, which indicates that the cells were being successfully seeded into the foam scaffolds. After perfusing the bioreactor overnight, the bioreactor cartridges were opened to collect the scaffold and the spheroid formation was assessed by staining the live cells with Calcein AM. Both the top and bottom of the scaffold were observed, as shown in FIG. 10. FIG. 10 shows the resulting spheroids formed one day after seeding at the top and bottom of both bioreactors. Spheroids were present in both scaffolds and throughout each scaffold, from the bottom to the top. Cell counts after scaffold digestion and spheroid dissociation, as shown in FIG. 9B, indicated a slight cell loss compared to initial cell seeding into the media stock bottle with a 90% cell recovery in reactor 1 and a 60% cell recovery in reactor 2 These data indicate that scaffold can be colonized by cells directly through perfusion of media carrying cells in suspension and that in the scaffold, these cells form spheroids under prefusion.

### Example 7

In this experiment, the optimal scaffold composition (pore and interconnect size) was assessed for hMSC spheroid formation and maintenance under perfusion up to 10 mL/min media flow rate for 48 hours. FIGS. 6A-6D show uncoated foam scaffold compositions (FMXP005 (FIG. 6D), XP64 (FIG. 6B), XP76 (FIG. 6C), and XP49 (FIG. 6A) into which hMSC cells were seeded inside of a perfusion bioreactor.

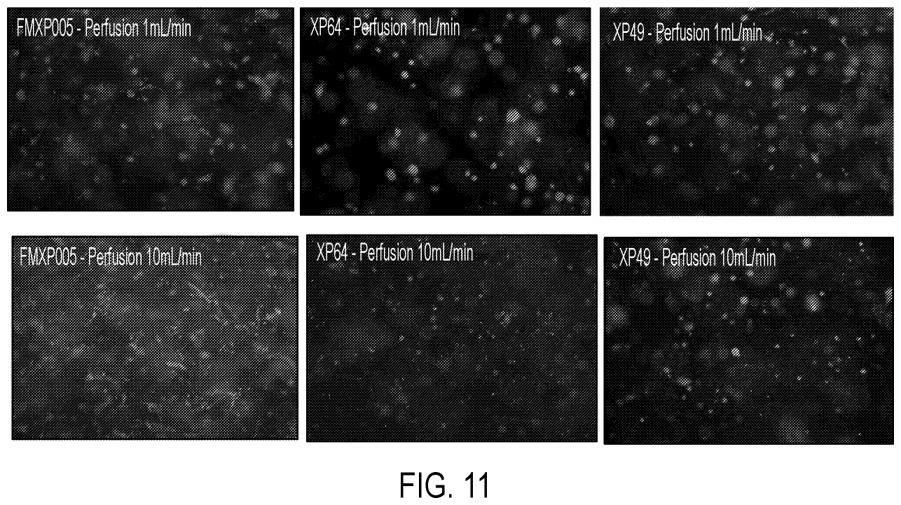

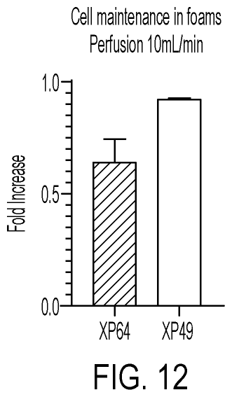

Cell seeding was performed as described in Example 5 for hMSC, but 75 μL of cell seeding solution containing 2×10⁶ cells/mL was seeded. After over-night spheroid formation, the scaffolds were transferred into bioreactor cartridges. In order to test possible perfusion flow rate dependence on the maintenance of spheroids, each foam scaffold was tested at perfusion flow rates of 1 mL/min and 10 mL/min. The first row in FIG. 11 shows the resulting spheroids present after 48 hours for the 1 mL/min perfused bioreactors. The second row in FIG. 11 shows the resulting spheroids present after 48 hours for the 10 mL/min perfused bioreactors. After 48 hours of perfusion in the bioreactors, the bioreactors were opened to collect the scaffolds, and spheroids or cells observed after Calcein AM staining. Interestingly, even without a cell adhesion coating on the FMXP005 foam scaffold (FIGS. 6D and 11), some hMSC adhesion on the scaffold was observed, especially at the 10 mL/min perfusion flow rate. This could be due to the specific composition of this scaffold or the increased shear force/turbulence in this scaffold at this higher flow rate due to the smaller interconnects or number of interconnect per pores. As such, the other compositions may be preferable for spheroid formation and EV production, at least when using hMSC. In addition, both the XP64 and XP49 foam scaffolds lead to good spheroid formation at the 1 mL/min perfusion flow rate, indicating that both scaffold composition can be suitable for spheroids formation. However, more pronounced spheroid and total cell loss was observed with the XP64 foam scaffold (compared to the XP49 foam scaffold) at the 10 mL/min perfusion flow rate, as shown in FIG. 12. This spheroid loss at 10 mL/min in the XP64 scaffold compared to the XP49 scaffold is most likely due to the higher number of interconnect per pores in the XP64 scaffold. These data may suggest that for this scaffold composition, higher perfusion flow rates may not be optimal for EV and other particle production by spheroid stimulation. In addition, these data indicate that a specific scaffold composition with pore and interconnect size may be preferable to maintain spheroids inside the scaffold at higher perfusion flow rates for certain applications. Accordingly, embodiments of this disclosure include tuning the pore and/or interconnect size depending on the cell type or desired cell byproduct.

### Example 8

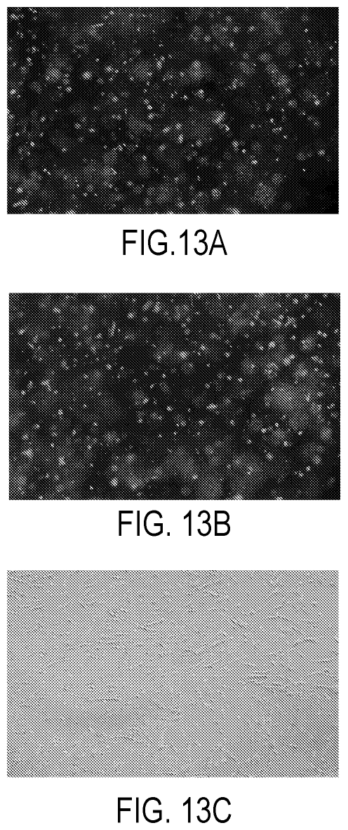

In this experiment, it is demonstrated that the foam scaffolds according to embodiments of this disclosure can be used to strongly increase extracellular vesicle (EV) and particles production from spheroids. For this example, only the XP49 scaffold composition was used. Cell seeding (with hMSC) was performed as in Example 6. After over-night spheroid formation, the scaffolds were washed with RoosterCollect™-EV media (defined, low particle media) from RoosterBio, Inc., to remove all remaining EVs and other particles present in the culture media. The scaffolds were then added into the cartridges of bioreactors. RoosterCollect™-EV media was added into the media stock bottle and the perfusion flow rate was adjusted to either 10 mL/min or 2 mL/min. For the 2D static condition, cells were washed with RoosterCollect™-EV media and then incubated in RoosterCollect™-EV media for 48 h in the static condition. After 48 hours, the bioreactors were opened to collect the scaffolds, and spheroids or cells were stained with Calcein AM. In this example, a similar number of spheroids were observed in scaffolds that were perfused at flow rates of 10 mL/min (FIG. 13A) and 2 mL/min (FIG. 13B). Cells presented a normal shape in the 2D static condition (FIG. 13C).

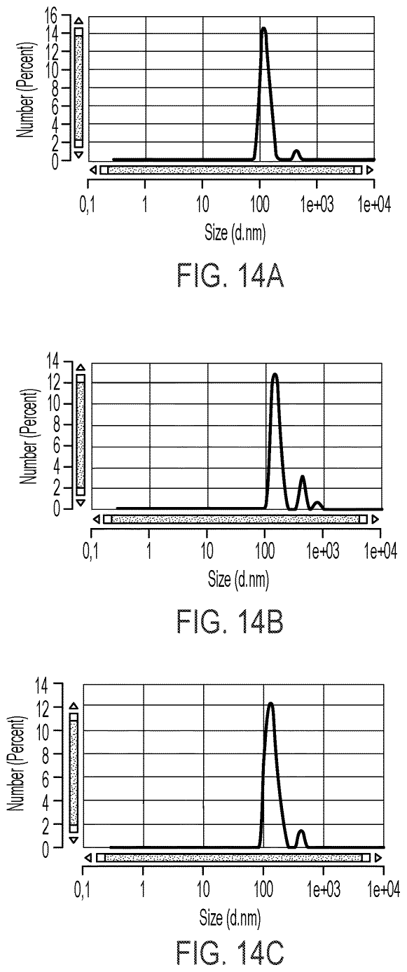

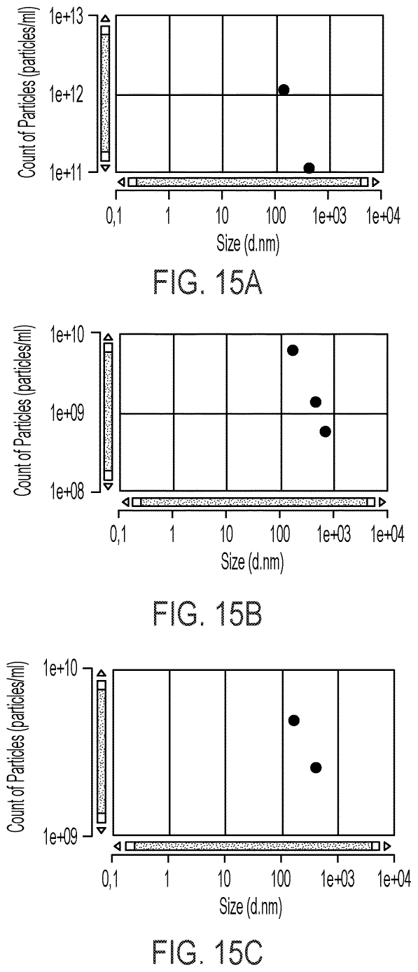

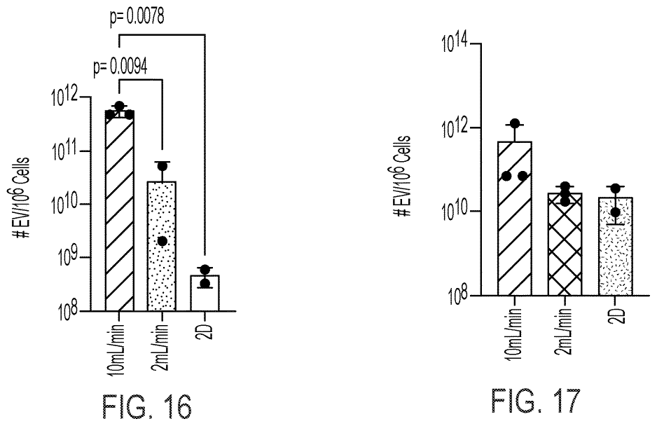

The conditioned RoosterCollect™-EV media was collected, processed to remove cell debris and large particles by centrifugation at 300×g for 15 min and filtration through a 200 nm filter. This clarified media was then concentrated using 100 kDa ultrafiltration membrane and EVs were isolated through over-night precipitation in ExoQuick-TC™ (System Biosciences) and centrifugation at 1500×g for 30 min. EVs were then resuspended in sterile dPBS. Each EV sample was then analyzed to assess particle size, concentration, and EV quality and functionality. Particle analysis by multi-angle dynamic light scattering (MADLS) was performed, and the results are shown in FIGS. 14A-14C, 15A-15C and 16. FIGS. 14A-14C show the size distribution of particles or EVs detected by number (which is proportional to particle abondance in the sample). FIGS. 15A-15C show the concentration of particles/EVs for the different images of particles detected. MALDS combines the scattering angle information from Mie theory and the particle size distribution analysis from a dynamic light scattering measurement in an integrated method. The lower noise, and hence reduced smoothing, enables a reliable and accurate representation of the particle size distribution with good characterization of individual components of a multi-component sample. Using the MADLS method, a population of extracellular vesicles having an average diameter of about 100 nm was identified, as shown in FIGS. 14A-14C and 15A-15C. This population was significantly more abundant in the perfusion sample at 10 mL/min (FIGS. 14A and 15A) than at the one at 2 mL/min (FIGS. 14B and 15B) or the one from the static culture condition (FIGS. 14C and 15C). FIG. 16 shows the number of EVs per million cells for each condition: 10 mL/min perfusion, 2 mL/min perfusion, and static 2D culture. As shown in FIG. 16, the perfused 3D scaffolds produced higher EV counts per million cells compared to the 2D growth condition after 48 hours.

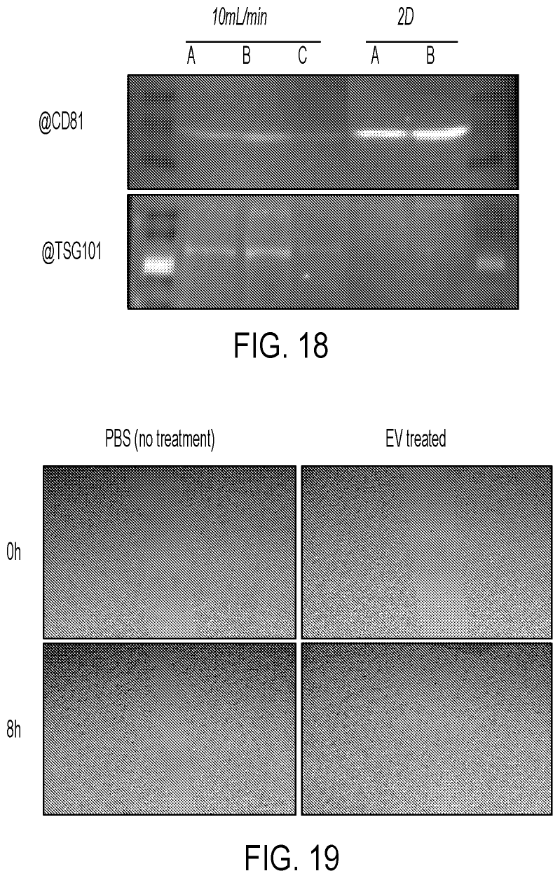

Similar results were observed by quantifying the CD63 positive EVs (a specific EV population) by enzyme-linked immunosorbent assay (ELISA), as shown in FIG. 17. The perfused foam scaffolds showed higher numbers of CD63 positive EVs per million cells compared to the 2D environment, with the higher perfusion rate of 10 mL/min having a significantly higher number of EVs per million cells. The presence of EVs in these samples was also confirmed by Western blot analysis against CD81 and TSG101 markers, two other markers of EV populations, as shown in FIG. 18. The blot analysis in FIG. 18 shows the results for CD81 and TSG101 for the 10 mL/min perfusion reactor and the foam used in a static 2D culture plate.

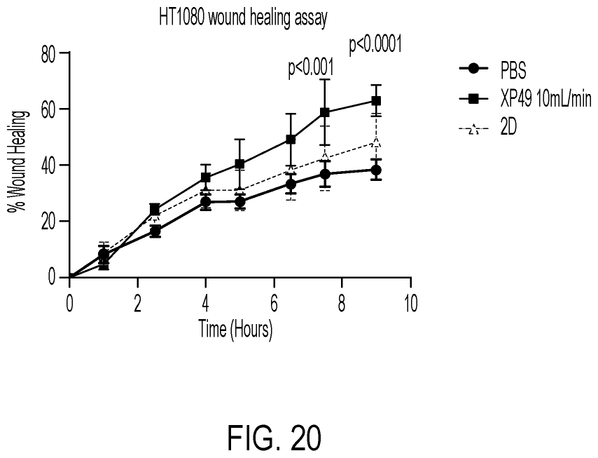

Next, the functionality of these EVs was assessed by a wound healing assay with HT-1080 cells. The wound-healing assay is a method to study directional cell migration in vitro. This method mimics cell migration during wound healing in vivo. The basic steps involve creating a “wound” in a cell monolayer, capturing the images at the beginning and either at a set time point or at regular intervals during cell migration to close the wound, and comparing the images to quantify the migration rate of the cells. After performing a wound on the tissue culture plate, wells were either treated with 2×10⁹ EVs from perfusion sample at 10 mL/min (using the XP49 foam scaffold) or from 2D samples, or were only treated with phosphate-buffered saline (PBS) as a control. FIG. 19 shows brightfield contrast phase microscopy images of the cells right after the wound was performed (Oh) for both a PBS-treated sample and an EV-treated sample, followed by a photograph of each sample after 8 hours. A significant increase in wound healing speed was observed when treated with the EVs from the 10 mL/min perfusion sample compared the PBS treated wells, as shown in FIG. 20.

In FIG. 20, the percent of wound healing over time is plotted for PBS-treated samples, samples treated with EVs from hMSC spheroids in foam scaffolds in perfused bioreactors (XP49, 10 mL/min flow rate), and samples treated with EVs from adherent hMSC spheroids on a 2D surface. The samples treated with EVs showed higher would healing percentages compared to the PBS-treated sample, and the samples treated with EVs from the perfused bioreactors showed the best wound healing by a significant margin. Thus, EVs from the perfusion sample are shown to be functional and even more efficient than the EVs from the 2D sample, as shown in FIG. 20. These data indicate that hMSC spheroids can be maintained in the scaffold through perfusion of a defined, low nutrient media like the RoosterCollect™-EV media. In addition, increasing the perfusion flow rate led to an increase in particles and EV production from these spheroids. The presence of EVs, as well as their quality and quantity, was confirmed.

### Example 9

This example compares EV production from hMSC spheroids in different culturing vessels with EV production by the scaffolds of the present disclosure. It determines the impact of shear stress on the hMSC spheroids' ability to produce quantities of functional EVs using perfusion with the scaffolds of the present disclosure, agitation, or classic 2D culture conditions (i.e., static conditions).

More specifically, scaffold composition XP49 was used to assess EV production from hMSC spheroids under perfusion with scaffolds of the present disclosure. This was then compared to EV production from hMSC spheroids in microcavity-containing vessels under agitation (35 rpm) and in T-75 CellBIND® flasks (Corning®) (used as a classic 2D static condition).

Cell seeding with hMSC was performed as in Examples 6 and 7 for scaffolds of the present disclosure. For the microcavity-containing vessels with agitation, cell seeding was performed following the manufacturers' recommendation. For the T-75 CellBIND® flask, around 9×10³ cells/cm² were seeded. All vessels and scaffolds were seeded at the same time. After leaving the vessels overnight for spheroid formation, scaffolds were washed with RoosterCollect™-EV media (defined, low particle media, RoosterBio M2001) to remove EVs and other particles present in the culture media.

For experiments using scaffolds, the scaffolds were then added into a bioreactor set up like the embodiment shown in FIG. 5B. RoosterCollect™-EV media was added into the media stock bottle and the perfusion flow rate adjusted to 10 mL/min. RoosterCollect™-EV media was also added to the microcavity-containing vessels with agitation and to the T-75 CellBIND® flasks. For the microcavity-containing vessels with agitation and the T-75 CellBIND® flasks, cells were washed with RoosterCollect™-EV media and then incubated in RoosterCollect™-EV media for 48 hours either under agitation at 35 rpm (microcavities vessel) or static conditions (T-75 CellBIND® flasks).

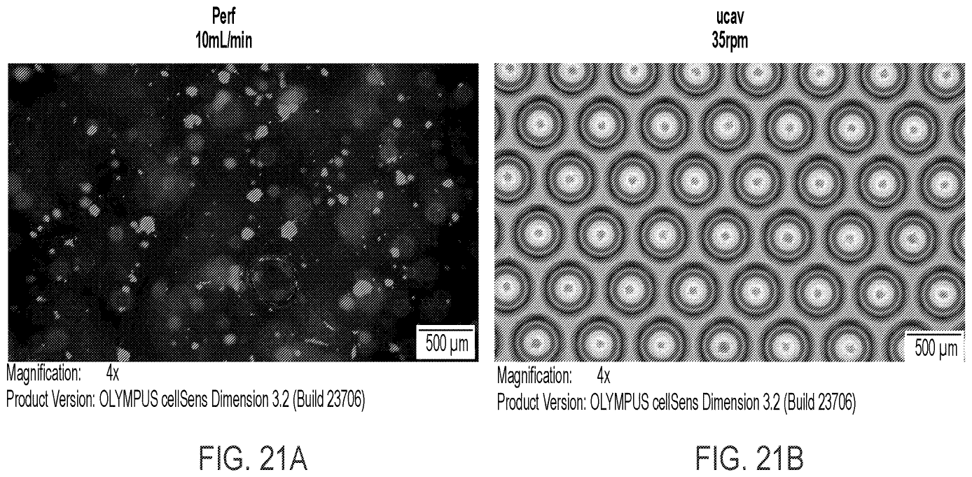

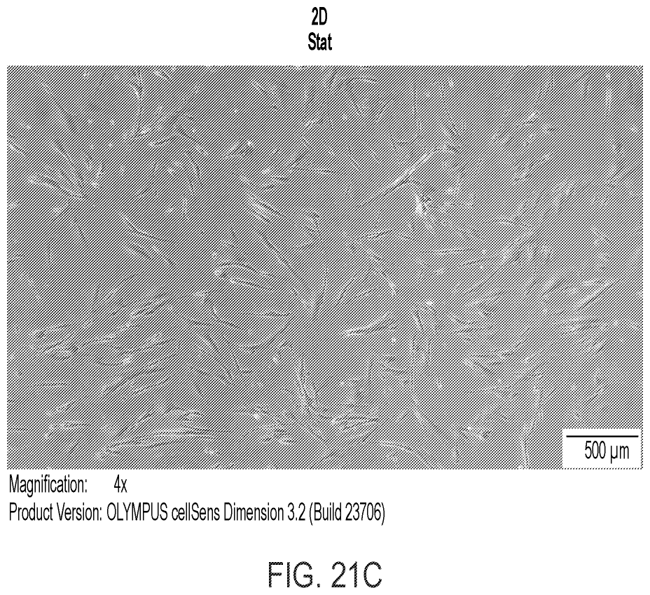

After 48 hours of perfusion, agitation, or static conditions, the bioreactors were opened to collect the scaffolds and spheroids, and the cells were stained with Calcein AM (results shown in FIGS. 21A-C). FIG. 21A shows spheroids in the scaffold after the perfusion conditions. FIG. 21B shows spheroids in the microcavity vessel after the agitation conditions. FIG. 21C shows spheroids in the T-75 CellBIND® flask after static conditions. Spheroids were observed in the scaffolds at 10 mL/min perfusion flow rate conditions, the microcavities vessel with agitation conditions, and in the 2D static condition.

The conditioned RoosterCollect™-EV media was collected and processed similarly to Example 8 to remove cell debris and large particles and was then concentrated and EVs isolated and resuspended in sterile Dulbecco's Phosphate Buffered Saline (dPBS). Each EV sample was then analyzed using the same techniques as Example 8 to assess particle size, concentration, EV quality and functionality.

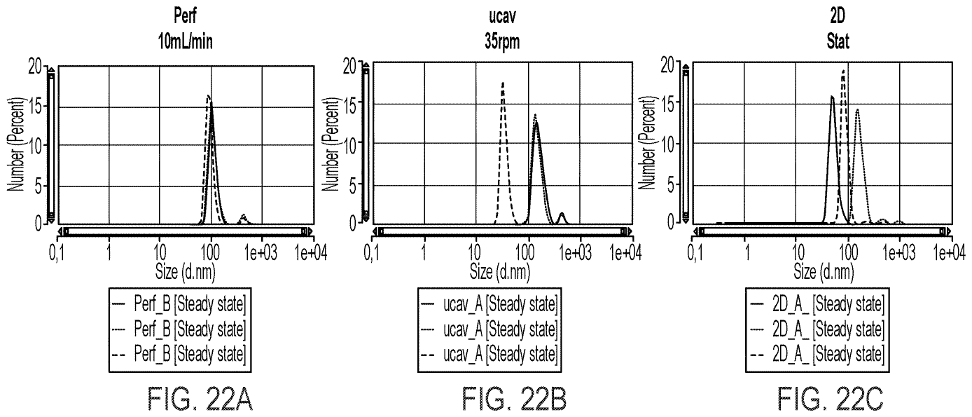

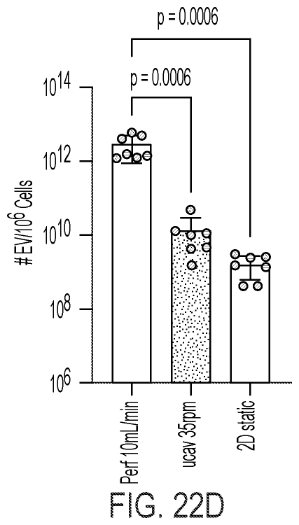

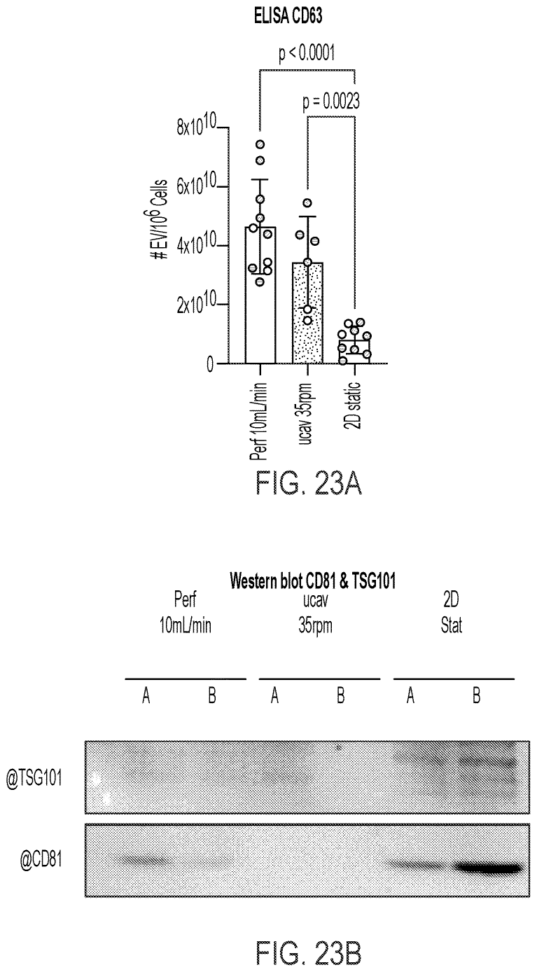

The MADLS method identified a population of about 100 nm size diameter (average) for all conditions (FIGS. 22A-C). FIGS. 22A-C show triplicate measurements of the same sample for each of the perfusion, agitation, and static condition experiments. This population of particles was significantly more abundant in the perfusion sample at 10 mL/min than in the microcavities vessel under agitation or the 2D static condition (FIG. 22D). An ELISA assay quantifying CD63 positive EVs in these samples indicated no significant difference between the perfusion samples and the microcavity under agitation samples. However, significantly more CD63 positive EVs were detected in the perfusion samples compared to the 2D static samples, as well as in the microcavity under agitation samples compared to the 2D static samples (FIG. 23A). The presence of EVs in these samples was also confirmed by Western blot against CD81 and TSG101 (FIG. 23B). FIGS. 23A-23B show data of ELISA CD63 and the Western Blot (using CD81 and TSG-101 markers) experiments with data from four independent experiments, where each dot represents one sample. Dots of the same color belong to the same experiment.

The functionality of these EVs was assessed by a wound healing assay with the HT-1080 cells that was performed as described in Example 8 and after generating a wound, each well was treated with 2×10⁹ EVs from the perfusion sample at 10 mL/min, the agitated microcavities sample, or the static conditions sample. A control was also performed where a wound was only treated with phosphate-buffered saline (has no EVs) as a negative control.

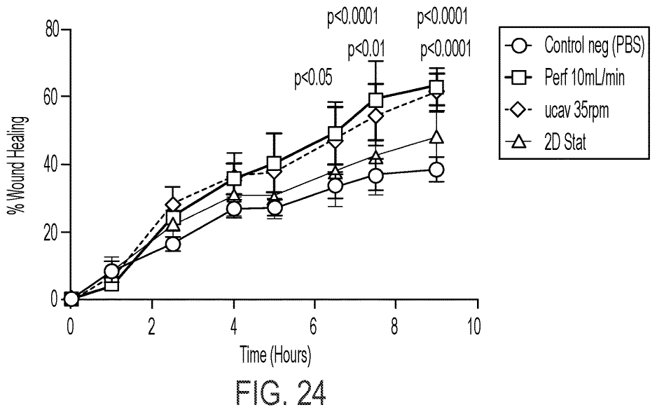

A significant increase in wound healing speed was observed with the EVs from hMSC spheroids. The EVs from the 10 mL/min perfusion samples and the agitated or static condition microcavities samples led to faster wound healing compared the PBS treated wells (FIG. 38). The EVs from the perfusion samples were slightly more efficient than the EVs from the static condition samples (FIG. 24).

The results indicate that hMSC spheroids under some shear stress produce significantly more EVs per cell than classical 2D culture. In addition, hMSC spheroids under perfusion produce significantly more particles than hMSC spheroids under agitation and 2D static conditions but not significantly more CD63 positive EVs compared to the microcavities conditions according to the ELISA data. Further, EVs from hMSC spheroids under perfusion or agitation conditions seem to be more efficient in the wound healing assay than EVs from 2D static condition.

### Example 10

This example compares EV production from hMSCs in the scaffold at higher and lower perfusion flow rates than previously assessed. The results provide lower and upper limits of perfusion flow rates that provide higher yields of EVs per cell in EV production.

In this experiment only scaffold composition XP49 was used to assess EV production from hMSC spheroids under perfusion. Similar to Examples 7 and 8, this scaffold was chosen because it presented the best spheroid maintenance under high perfusion flow rate compared to the other scaffolds previously tested. As a control, EVs were also produced from hMSCs adhered on T-75 CellBIND® flasks in a 2D static condition.

Cell seeding using hMSCs was performed as in Examples 7 and 8 for the scaffold. For the T-75 CellBIND® flask, around 9×10³ cells/cm² were seeded. All vessels and scaffolds were seeded at same the time. After over-night spheroid formation, scaffolds were washed with RoosterCollect™-EV media (defined, low particle media, RoosterBio M2001) to remove all remaining EVs and other particles present in the culture media. Scaffolds were then added into the cartridge of bioreactors. RoosterCollect™-EV media was added into the media stock bottle and perfusion flow rate adjusted to the different flow rates tested. Here EV production was assessed at 20, 10, 2 and 0.5 mL/min. EV collect media was also added to the T-75 CellBIND® flasks after two washes with RoosterCollect™-EV media.

After 48 h of perfusion, EV production was assessed. Bioreactors were opened to collect the scaffolds and spheroids were stained with Calcein AM. For the 2D condition, cells were observed without further staining.

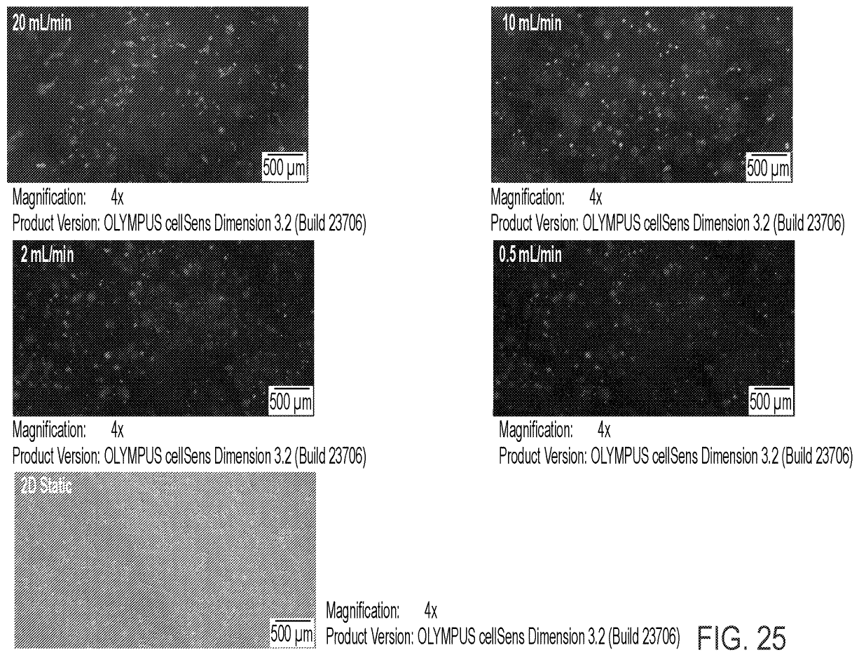

As previously observed, no strong difference in spheroid shape or density into the scaffold was observed between the 10 mL/min and the 2 mL/min perfusion condition (FIG. 25). Similarly, no difference was observed with the 0.5 mL/min perfusion condition. However, in the 20 mL/min perfusion condition, almost half of the spheroids appeared to have lost their morphology and appeared to either adhere or be in contact with the scaffold (FIG. 25). As was observed in the previous Examples, hMSCs in the 2D condition presented the expected spindle-like morphology (FIG. 25).

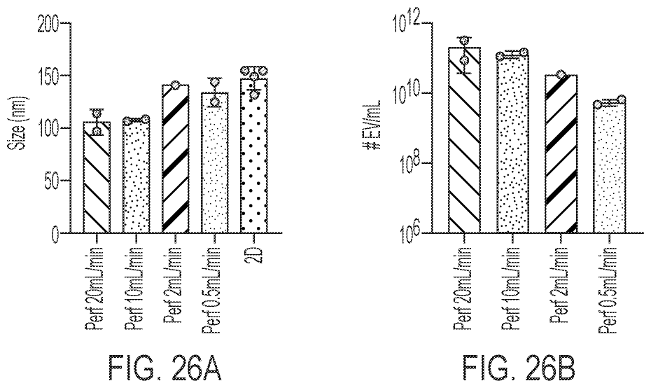

Apart from the spheroid and cell morphology observations, the conditioned RoosterCollect™-EV media was collected. To clarify the media, the collected conditioned media was centrifuged at 300×g for 15 min to remove remaining cells and debris and then filtrated though a 200 nm filter to further remove smaller cell debris and large particles. For this experiment, the clarified media was also assessed by MADLS to determine the size and concentration of EVs produced by hSMCs. For all conditions, the EVs had an average diameter of about 100 nm (FIG. 26A). Regarding particle concentration, strong differences were observed between the different conditions with up to around 2×10¹¹ particles/mL in the 20 mL/min perfusion condition and around 1.23×10¹¹ particles/mL in the 10 mL/min perfusion condition (FIG. 26B). These numbers than decreased in the other conditions, with only around 3.28×10¹⁰ particles/mL in the 2 mL/min perfusion condition and 5.38×10⁹ particles/mL in the 0.5 mL/min perfusion condition. However, no concentration was able to be calculated for the 2D condition, as the concentration of these particles was below the threshold of the equipment.

This clarified media was then ultra-filtrated to concentrate EVs and EVs were then precipitated over-night in ExoQuick® as previously described. The next day, all these samples were centrifuged at 1500×g for 30 min and EVs were resuspended in sterile PBS.

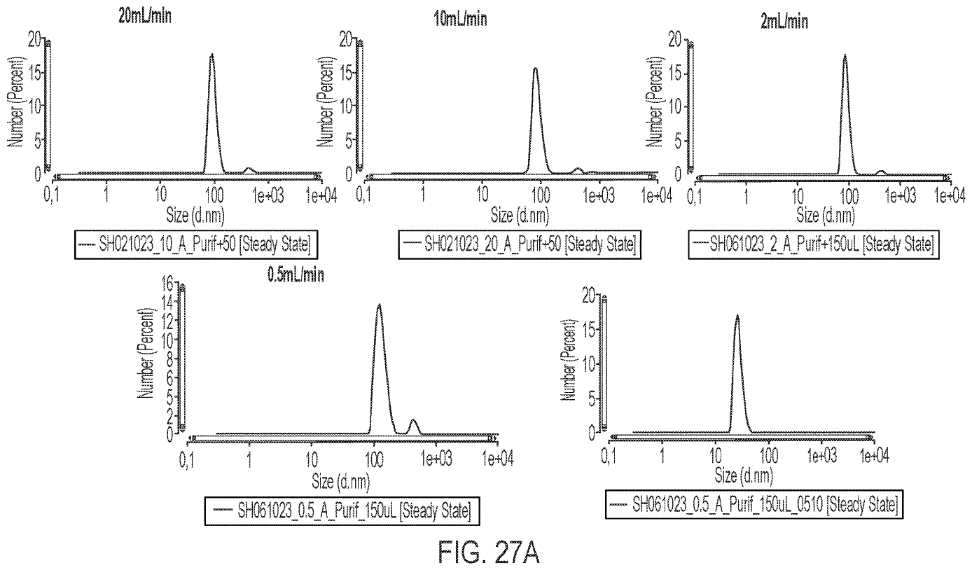

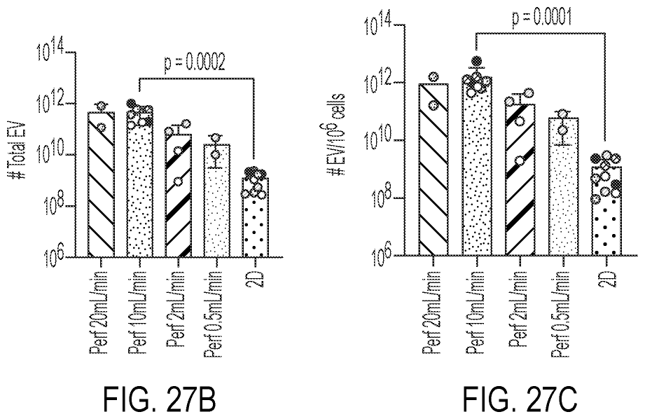

These purified EVs were again analyzed by MADLS to determine their size and concentration. As in the clarified media, the EVs had an average diameter of about 100 nm (FIG. 27A). Regarding the amount of EVs produced, no strong difference was observed between the 20 mL/min and the 10 mL/min perfusion condition (FIG. 27B). However, when compared to the other conditions, in the 2 mL/min perfusion condition there were about 7× less EVs than in the 10 or 20 mL/min perfusion condition, and in the 0.5 mL/min perfusion condition there were more than 10× less EVs compared to these higher flow rate conditions (FIG. 27B). More interestingly, there were about 100× less EVs produced in the 2D condition compared to these higher flow rate conditions (FIG. 27B).

For the amount of EVs produced per the number of cells for each condition (FIG. 27C), similar results were observed with the most number of EVs produced in the 20 and 10 mL/min perfusion condition and the lowest EVs produced in the 2D condition but with 1000× less EVs per million cells in the 2D condition compared to the 10 mL/min perfusion condition.

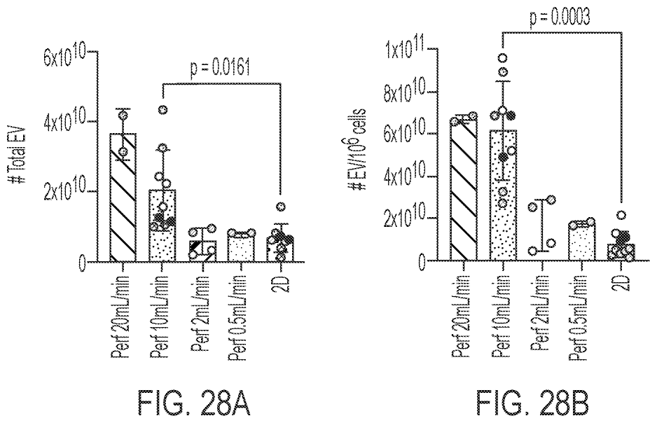

Next, these samples were further analyzed to determine the amount of CD63 positive EVs by ELISA. Regarding the total CD63 positive EVs (FIG. 28A), the highest level of CD63 positive EVs was observed in the 20 mL/min and the 10 mL/min perfusion condition. No strong difference in CD63 positive EVs was observed between the lowest perfusion flow rates at 2 and 0.5 mL/min and the 2D condition (FIG. 28A). However, the total CD63 positive EVs per million cells for each condition (FIG. 28B) shows a slight increase of about 2× more CD63 positive EVs in these lowest perfusion flow rates compared to the 2D condition.

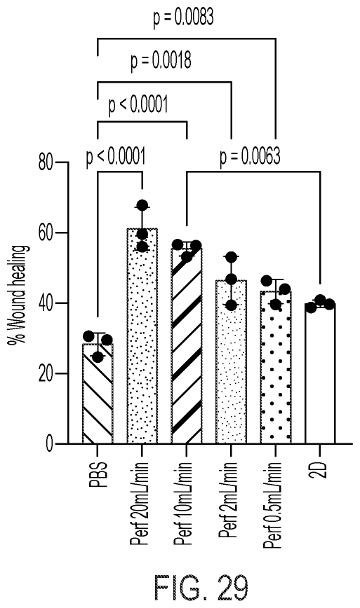

The functionality of these EVs was also assessed by the wound healing assay with the HT-1080 cells (FIG. 29). Here this assay was performed slightly differently from before. The wound was created by a P1000 pipet tip and wound healing assessed only at 6 hours post-injury. Each wound (other than the control), was treated with 2×10⁹ EVs from each of the conditions. The control was treated only with PBS. At Oh and 6 h post-injury, images were taken from each wound and the distance between the edges was measured using the OlyVIA® software (Olympus LS). At 6 h, the distance between the two edges was compared to the distance at Oh to calculate a percentage of closure of the wound or wound healing. All perfusion conditions presented a faster wound healing than the mock control (FIG. 29). In addition, here it seems that increasing the perfusion flow rate led to a faster wound healing and also compared to the 2D condition, suggesting that other EVs beside CD63 positive EVs may play a role in the wound healing speed.

Taken together, the data indicate that increasing the flow rates in the perfusion samples leads to higher EVs production. However, the upper limit appears to be around 10-20 mL/min while the lower limit is even less than around 0.5 mL/min, as even at this low flow rate there is an increase of EV production compared to the 2D condition. In addition, the EVs produced by perfusion, even at higher flow rates such as 20 mL/min, are functional and improve wound healing speed compared to the control mock treated condition.

While the present disclosure includes a limited number of embodiments, those skilled in the art, having benefit of this disclosure, will appreciate that other embodiments can be devised which do not depart from the scope of the present disclosure.

1. A method for producing three-dimensional cell cultures or byproducts of three-dimensional cell cultures, comprising:
providing a bioreactor comprising a cavity for culturing cells, an inlet and outlet to the cavity, and a porous scaffold;
inserting cells of a cell type into the porous scaffold to form three-dimensional cell cultures; and
perfusing a cell culture medium through the cavity to culture the three-dimensional cell cultures;
wherein the porous scaffold comprises pores, and passages between the pores;
wherein the porous scaffold is non-adherent to the cells when perfused at a linear velocity from about 1.1×10⁻⁵ m/s to about 5.0×10⁻⁴ m/s; and
wherein at least 80% of the cells in the pores of the porous scaffold aggregate to form spheroids.

2. (canceled)

3. (canceled)

4. (canceled)

5. (canceled)

6. (canceled)

7. The method of claim 1 further comprising harvesting at least one of the three-dimensional cell cultures or at least one byproduct of the three-dimensional cell cultures.

8. The method of claim 7 wherein the byproduct of the three-dimensional cell cultures that is harvested is an extracellular vesicle.

9. (canceled)

10. The method of claim 1, wherein the porous scaffold comprises an ionotropically crosslinked alginic acid or salts thereof, or an ionotropically crosslinked polygalacturonic acid compound selected from at least one of: pectic acid; partially esterified pectic acid, partially amidated pectic acid, and salts thereof.

11. The method of claim 1, wherein at least 70% of the pores have a pore diameter from about 200 μm to about 1000 μm, when measured in a dried scaffold, wherein at least 70% of the passages have a maximum passage width from about 30 μm to about 500 μm, when measured in a dried scaffold, and wherein at least 70% of the pores have between 5 and 18 passages per pore.

12. (canceled)

13. (canceled)

14. (canceled)

15. (canceled)

16. (canceled)

17. (canceled)

18. The method of claim 1, wherein the porous scaffold is dissolvable by a non-proteolytic enzyme or a non-proteolytic enzyme and a chelating agent.

19. (canceled)

20. (canceled)

21. (canceled)

22. (canceled)

23. (canceled)

24. (canceled)

25. (canceled)

26. A bioreactor, comprising:
a cavity for culturing three-dimensional cell cultures from cells of a cell type or byproducts of such three-dimensional cell cultures;
an inlet and an outlet to the cavity; and
a porous scaffold in the cavity;
wherein the porous scaffold comprises pores and passages between pores;
wherein at least 75% of the pores have a pore diameter from about 200 μm to about 1000 μm, when measured in a dried scaffold;
wherein at least 75% of the passages have a maximum passage width from about 30 μm to about 500 μm, when measured in a dried scaffold; and
wherein at least 75% of the pores have between 5 and 18 passages per pore;
wherein the porous scaffold is non-adherent to cells;
wherein the porous scaffold is configured to contain three-dimensional cell cultures; and
wherein the byproducts of three-dimensional cell cultures comprise extracellular vesicles.

27. (canceled)

28. (canceled)

29. (canceled)

30. The bioreactor of claim 26, wherein the porous scaffold is dissolvable.

31. The bioreactor of claim 30, wherein the dissolvable porous scaffold is a foam scaffold.

32. (canceled)

33. The bioreactor of claim 26, wherein the porous scaffold has a composition comprising:
an ionotropically crosslinked polysaccharide is chosen from alginic acid and salts thereof, pectic acid and salts thereof, partly esterified pectic acid and salts thereof, partly amidated pectic acid and salts thereof, or a combination thereof.

34. The bioreactor of claim 33,
wherein the ionotropically crosslinked polysaccharide is polygalacturonic acid.

35. (canceled)

36. (canceled)

37. (canceled)

38. A perfusion bioreactor, comprising:
a cavity for culturing cells;
an inlet and outlet to the cavity; and
a porous scaffold in the cavity, wherein the porous scaffold is non-adherent to cells;
wherein the porous scaffold comprises pores and passages between the pores,
wherein at least 75% of the pores have a pore diameter from about 200 μm to about 1000 μm, when measured in a dried scaffold,
wherein at least 75% of the passages have a maximum width from about 30 μm to about 500 μm, when measured in a dried scaffold,
wherein at least 75% of the pores have between 5 and 18 passages per pore; and
wherein the perfusion bioreactor is for culturing three-dimensional cell cultures or byproducts of three-dimensional cell cultures.

39. (canceled)

40. The perfusion bioreactor of claim 38, wherein the bioreactor is configured to retain at least 60% of any cells that are added through the inlet to the cavity.

41. The perfusion bioreactor of claim 38, wherein the bioreactor is for producing extracellular vesicles.

42. The perfusion bioreactor of claim 41, wherein wound healing by the extracellular vesicles produced by the perfusion bioreactor is greater than wound healing by extracellular vesicles produced in a 2D flask for 3D cultures, at 6 hours post-injury, when measured by a wound healing assay with HT-1080 cells.

43. The perfusion bioreactor of claim 41, wherein wound healing with about 2×10⁹ extracellular vesicles produced by the perfusion bioreactor has at least 5% better wound closure than wound healing with 2×10⁹ extracellular vesicles produced in a 2D flask for 3D cultures, when measured by a wound healing assay with HT-1080 cells at 6 hours post-injury.

44. The perfusion bioreactor of claim 41, wherein the extracellular vesicles produced by the perfusion bioreactor are produced in an amount between about 1×10³ EVs per cell and about 1×10⁷ EVs per cell.

45. The perfusion bioreactor of claim 44, wherein the extracellular vesicles are produced from perfusion with a linear velocity between about 1.1×10⁻⁵ m/s and about 5.0×10⁻⁴ m/s.

46. The perfusion bioreactor of claim 44, wherein an increase in perfusion flowrate increases the number of extracellular vesicles produced per cell.

47. The perfusion bioreactor of claim 44, wherein the number of extracellular vesicles produced per cell with the perfusion bioreactor is greater than the number of extracellular vesicles produced with a static 2D microwell plate.

48. (canceled)

49. (canceled)
